SEQUENCE LISTING (30 sequences)

SEQ ID NO: 1 - DNA, 4428 bp, Mus musculus
aagttcctgctgagcagatcagcctaatgcttaaatagaacaactcctggctgtcattgacattgtctaaaagccaagatgacagactgagaggcctgagcccttgttctggcattctcccaggaagatgcagtaaaggggttgacccaatatactgcagagaatttcatccagttccctcctccatcctgatcccatgtgccaggaagatggagccacgcttgctgatgttggggtttctctcattaaccatagtacccagttgtcgggcagaactgtgtctgtatgacccacccgaggtccccaatgccacattcaaagccctctcctacaagaacggcaccatcctaaactgtgaatgcaagagaggtttccgaagactaaaggaattggtctatatgcgttgcttaggaaactcctggagcagcaactgccagtgcaccagcaactcccatgacaaatcgagaaagcaagttacagctcaacttgaacaccagaaagagcaacaaaccacaacagacatgcagaagccaacacagtctatgcaccaagagaaccttacaggtcactgcagggagccacctccttggaaacatgaagattccaagagaatctatcatttcgtggaaggacagagtgttcactacgagtgtattccgggatacaaggctctacagagaggtcctgctattagcatctgcaagatgaagtgtgggaaaacggggtggactcagccccagctcacatgtgtagatgaaagagaacaccaccgatttctggctagtgaggaatctcaaggaagcagaaattcttctcccgagagtgagacttcctgccccataaccaccacagacttcccacaacccacagaaacaactgcaatgacggagacatttgtgctcacaatggagtataaggtagcagtggccagctgcctcttcctgctcatcagcatcctcctcctgagcgggctcacctggcaacacagatggaggaagagcagaagaaccatctagcaagctagaaaagtcagagcccaggcaagcggatgggaatcacaaagctcaagccaaatctgagacgccaagcattcacctaacggctgtttccttctgatccctgggtttctagaacattctgaagtcacaggacataacagcaactctatcactaaactggactttgccattgaagaataggatctaaccacttcagcacagcagttctaaagctttaatgggagagagggcccaacagtgctctgtgtgttttgtttttgtgtatatctgttgatgggagctgagatggtgtggtcacttttcatgtaacatatagtatagaaaaagtagctttaggttgacttcattgttacaacccagtttggaaagcccaagtaaaactcagcactaatgtaaataattcctcctcctcctcctctctcttttcatcctccgctccatcttcctcttcttcctcctccttttccacctcctctgtccctacccacccccacccatccactttccttcttcctttctgctctcacaagctcatcctagctacacgtgcatggctggctcctttttcaacctctgtttgcctaactggctcttctgatttcatcacttactgatcagcctttaaaactctgagctggcaaagatgactctatctatgttcttggctcagtcccagaaggaaacccccttttcatgaagcttcagttttgacatcctgaagaacagaaactgtggcagaacaatcttcagataacatcaaaacaaagtggagaagccacgggaactgtggagctctggtattcagaagcctgtgtctagggtctgcgccaggagcagaaggctgaaggaagtcccaggacgtggacttagatgctttcccagcaggccactctaagcgctggtttctttgggacagctgtcaattgtacgctcaatttagcctgcactaatctgatgcttacaggtgaacactcaaggcacaggtatggacttggtacataccgtgaaaacactggaaagaaaagaatactttcaagtttacagaaggaaggaaggaaaaaggaagcagaggtggtgattatacaaaagattagctgtagactggatatcccaggcatcctcggataatgcccccgccccagcaccctgatccaggtcaccaaagccttgtgagatcagactgcagagccagtctgtctctgagtcagtaaatgtagaatttggatttctcacaagttcctggcggtgtcttttttttttttaatattttttattaggtattttcctcatttacatttccaatgctatcccaaaagtcccccataccctcccccacactcccctacccacccactcccactttttggccctggtgttcccctgtactggggcatataaagtttgcaagtcctggtggtgtctttatgctgatctctagcccacactttgtgaggcactgggctatcccagtgtgctctcctcttccacagataccaaaagcacctgggtttgatgctcagacttctgagcacgttcttgttcaatctcttgcgtaagatttcctctcagatgagttgagtcagattctcatgtttaacagtgttttaggggattcacagaagcccaaactatcagttttcatttctgaaaaggctggaaaattttatgaaaaactttcaaaggtcagacagagccattttgagtcttttatgtgaccaagtatgaaccagatctttcctatctatggtctcccctttccaaaatatatcttttgtggggacacggcaaggaggaaagttaaatagaatctcaagctactaattttagaaaagaaaaaaatattaaactcttactaaagagctgtgggtagtggtacacacctgtaagcccagctctcaggagactgaggcaggaggattgcagtgagtcagagatcagcttcacctacaagcaaaaccctgccttacccctcaacctttccataaaaacagtcttacttgtgtaaaattaatttttaatacatatttgtgcacgatgtgtgtgcctggtgcccagggaagccaaaaaaatatgttcaatgtccctggaataggtggttatgagctgccatggaggtgatgggatttaaacccctgttctctgaaaaagcagctagttctcttaaccactgagccatctctacagccccattaaattgaattttattgtcattactcaatatgggagatggggtaatgataacaattttttttttataatactaagatgtttagctattttactctccttcacaagtgtagagtagaattttctagaagctacatgcatgatattatcgctctggtgttaacacataatggatttatcttgttaataaaagaattagtaaataatattttaaattttttctttgttttagttttaagatgattaatatctatagatactagtgtacattaagaaagcctttgggatcctcaatcattttgcatggttttagtaattttttaataacataaagaaggtctgacagattatgctaaagagctattgtggtatggattagaaatggcccccacaggctcctgtgttcaaacatcagctagcagtgctgctttgggttttggaacctttaagaggtggggccttgcttaaggagataggtcactggaggtgaaccagacttgcttccagtctccctctctgctgtcccaggaagccatcatatgaggagtttcacaacatacttctgctgccacagagtccctccagtaaggcacgtactggccactgtgtagtgtaccctctaaaactgagctagagctcccctctcctccctacactatctctgttgatgctttgtcacggtgatgaagaacataagtaaggcaaagacaagacatagtttggagactcacgtgagcatctcagccagactcaggcacagctgcgatgtgggaattatcaagcatgagatgcaaagcaatggaaaatgaatcgttatgacagaagcctacatctagtcttcccttcttcccattagtaataatagcccgtgttttagaagaacacatctttttggtgttctaggtagcttatattgcaaatgtggcacaatctaagagaaatctgggatgagggaacctcagtgaaagattctccttgatcagattgtcccactgatgattgatatgggaaggcccaacccactgtgaaaggcaccacccattggcaggatgactggggttatagaagcaaacaggctgagcatgagccagtgagcaagccagtaagcagtggcttctctttggaagaagcatggctttcttccgtggcttctgtttgatctctctcaatgatagattgtgacctagaagtataagctaaaataaaccatttcttacccata

SEQ ID NO: 2 - protein, 268 aa, Mus musculus
MEPRLLMLGFLSLTIVPSCRAELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKVAVASCLFLLISILLLSGLTWQHRWRKSRRTI

SEQ ID NO: 3 - DNA, 3216 bp, Homo sapiens
ggcagtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagatgagcataccagcagtccttgaacattatcaaaaagttcagcatattagaatcaccgggaggccttgttaaaagagttcgctgggcccatcttcagagtctctgagttgttggtctggaatagagccaaatgttttgtgtgtctaacaattcccaggtgctgttgctgctgctactattccaggaacacactttgagaaccattgtgttattgctctgcacgcccacccactctcaactcccacgaaaaaaatcaacttccagagctaagatttcggtggaagtcctggttccatatctggtgcaagatctcccctcacgaatcagttgagtcaacattctagctcaacaacatcacacgattaacattaacgaaaattattcatttgggaaactatcagccagttttcacttctgaaggggcaggagagtgttatgagaaatcacggcagttttcagcagggtccagattcagattaaataactattttctgtcatttctgtgaccaaccacatacaaacagactcatctgtgcactctccccctcccccttcaggtatatgttttctgagtaaagttgaaaagaatctcagaccagaaaatatagatatatatttaaatcttacttgagtagaactgattacgacttttgggtgttgaggggtctataagatcaaaacttttccatgataatactaagatgttatcgaccatttatctgtccttctctcaaaagtgtatggtggaattttccagaagctatgtgatacgtgatgatgtcatcactctgctgttaacatataataaatttattgctattgtttataaaagaataaatgatatttttt

SEQ ID NO: 4 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 5 - DNA, 3886 bp, Artificial Sequence
  synthetic nucleic acid sequence
ttcttaacagcttggtgagtgccacttcagtgagtgtgtgtcttcaataatcacaatattttctttggaaacatgactattcgagtgtttgttcatctatcgattcagttgccctttagtgttgctatgtaggtttttgtgtattctggaaacaaatattgtcatatatacattataaatggttttctcacagcctgccacatctcctccattttctcatggtctttggattagctgatacttttgtaattttttttttcttttataaaatctttaacctggagcagggcatgggagggtggactcagaccctctgatgctggggttacaggtgcttgtgagctgcttggttcaggtggctgggaagcgaacattggtcctctgcaaaggtagcaagcattctcagcaactgagccatctctccagcccccttttccttttatattcagtgcatgctctgtcctaagaaatcttcacctatccacaacttctaaagctgtctgcctttatttccttttagaagttttacagttttaattctatgattgcatgtgatacacacacacacacacacacacacacacacacacacacacacacgttcttattatggaagagaaataagttttctctgtggaaggaagctcttttcccttttcacatttatccacacgactcaagagtatttgttgagaacgttgtcctcttttcatgaattgctttggtaaatgggggggggggcagtaattaaatgttcctgagtttctagaatgtatattctgtcgctgtacttgtttttctcccagttgcttgctgtcttgatggctagctttatagtaacagatgttatgagtctcctatcatacactttatcttacagttatcttagatacttccaaggaagtttgaaaacaattttccaattttactaatatgtacatcaaaccagtgaattgaaggtaaactgacatgtcttttaaaaatcagccctccaatttataaacagtgtatgtttctctgtttatttcacttaaaaattttttctcagcagaaattcttttgcaattttcaagtctgttcttgtaggtgtgggtgaatttattcttgagtattttatgattctgatgtgtaattacaattaactttagatcttaattaattcattcagtttaaatatataacaagtgatttgagatagccacacctacctacttacatacgccattatgtagaattctcggtactttcatggaaagaggcatttttagctgtgttaagtgccctttcccacaaaactactttctgatagtaacatagcccatatcagctttcctatgcttatctctgcatatctttttctattctttcactcaattcattttggtttttatattttagcattttctttgtgagatagtatgcagttgttgtggtctgaaagtttctattttagaaaaaattgttcatttatgtttactataatcactagtagtgggttgacgttgatgtctgccttcttgctctttcttcgtgcttcttcatttcacctaaacagatctgtttaatacatattggaatgttctgtttaatttattctgttatcttttaatgtgtgctcctggtagtatttcattattgggtgccttccctggaatatttactatgcattcttaattttctatgttctatttagttagcattgtaccatttcagacatgctagaaaaatttaaaaagctaaaggcccatcaacctcagctttcttgcccaagttgtcacgttttacatttaagtaggtactaagatctgtcctgtagggaccagcacggtggctcagtggctaagaatgcatgcacactgctcttccagaagatctgagtttagttccaggcactcacactgtacagctcacaatcacctgtttctgacgccctcttctggtctctaggagaactgcactcaccccctcccctaacacacacagttaaatcaaaaataaataaaagacctaccatacagtgctattttttttttattttagacatctctttcaagaaaaaactggaattttctcatgaatatatacttactaatgcccttcattattcccagtagatttctatttttgactaatataatttccctgggtcttgacattttgtccttagctttcttttcacttttaatatgcactataagtgtgctagtggggttttgtgaactttcttttacctgaaaatgtgctaaccccatcctcatttttgaagggtgtcacgtctaatcaggattgagctgaaagcctgtctgtgtgtctgtttcgaggctggctctgtttccctgttattttctggctccctttgctctgtggtgaagtcagttgtcatccatcactgttcccactaatgtgtgtggcctttccctctggctcctttaagacattctctttagatttttggcagcttggcattagatgcataacaatttgcttttaaaagtctactctggtgtcatgaacatcttggatctgtaagttaacatctgtcttcaagagtcagaaatgagctggcaagatgggtaagtgtgtacaggtgtttcctattggtggccctcccacatttcatccttcccaaacagttccaccaagcaggaccaagcattcaaatatataagtctatgagggacattttctttcgaaccactaaacagagggatagaaaagggggcggggactggatgtgtgggttggggtgatagatagaacatacttctttgccttctacttcctgtcccagctacatctccagctgactcttgggtgcctgcccacactgagggcggggtcttttccctattattttgctgacctgtagtcagtcttctcaggcaatgtgcttacagatacacccagaagtatgtttcaaattctctaggcatccattagagccttcagtcgacacctcaaactagccgtcatgatactcaccagtctaatgtataagcaggtgcagtgttggcctcggtgttggcctcagcatgccgttccagatggagcaggtgggcaggatggcagcttgattacaaccaccttcacacactcagccatcatgccatgacagccccctgctttcctgccactcccactccttaccatcactcactcccccctccaccccccacctgctgtggtagtctcctagtcacctgatgtcttcaagcacaagagcttgttgcaatggaatggttttcacttacatcagggtccctcttcccttcaagagtcgggcatagagagctggctgtggcagtgtgcacctgtaatcgaaagacttgggactgaaagcaggatggtaggagttcatcctcagctacataacgaacttgagcccaattgggcctacaggagaacactccctctcaaaatgattacatgggaacctctatttctggtgatcatgctaggacacctgacctgtctcagaagctttggcccacccaacctctctcctgtgtatgctagacacaaagtaccttcagagtgaaattctacttatttgtacttattccttctacagtttgcattttatactgtagaggaacttgagtgaacactcctatagcatctctgacacaaatatttatctcttcagttttatacaatgggtttcattgtgacttttccatagatgcatataatgtattttgatcatattcacacacacacacacatacacacacatacacacatatcatcctctgtgctcccactgatcccgaggaggtagagtacgtaccaatcctttaaattgagagaagaagcatgtgagcaagctactgcacaatcaaactcatgtctcaccaacattctccttgcctttgcagaactgtgt

SEQ ID NO: 6 - DNA, 5776 bp, Artificial Sequence
  synthetic nucleic acid sequence
gccagaacgacatagtgagaccttgtttcaaagaaataaaagaatttttaaaaagagaaagataaatgtttgttcccagcttactctcctttttatggaacctatggaatggggccccacatttagagcagtctccctacccatttagcctaatcttgataacctctcaaggacgtgtccagaacgttatctctaatgtgactcgaaaacctgtcaatcaacataaaccaagagcatcatcagagttaaatcacccaaaaccagtttctcccacatacattttcaagaacaatgcctcccaaactttggtgagagacaaagcaaggatccagggagagaaaatatagtgtccatacgcacctcagttattcctggtaggaggcaaaagcaggccttcctgaaccacggaatcaatggaagcttctggaagattcacatgtcagcatttgggaagtgtgagcacttccaggcgatatcggtgtgtttaggatttcttatcttgtttcaatagttcctttatgaaggatgatgctgctaatgaggactatcctctgggccttcctcaggttgagctgttctgtcaagtaccagcctctttctatggctgttccttctatgccccctctgctctttgaaggctccttgatccttctaagaaatacccagggagaagaggaaagaaagaagaaactgcaggccccagctgttcaggaggtccttgatcacctacagaattttctcacctgatctacagatccaacccccgatccatgcattcccaaagcatggtcctgacctgaactccatctccctctcttgacagtggccagctgcctcttcctgctcatcagcatcctcctcctgagcgggctcacctggcaacacagatggtaagtccaacactggaaggacccagcaccccagtgtctggtccctggcctggcatcgtgaacattgcctagagcttgacctcagctgacacgctgggaagatggctccgacgaaagcatgagatgggtgtgcaattcctaaaagctttgtttaaaaaaaaatcgaatgtgatgatggcatttgcctataattgtataattcaagagctgaggaggcaggcagaggtggatccctcatactctctggccagccagcctagagaaactggtgaactccaggttgatcaggagactctatttcaaaagtaatgtgaataatatactaaggaacaagattccaggttgacctctagactccacagtcatttactccctgacacacgtactcttactgaatgtgtatgcccggtactgagtgggttacattagcaaatgtcacccctactctcagaggtctgtgctgtttagagattaggaaaacctaggcttcccgaatgtgcagatagcaagagtttgaatcctatttagagatcttcaaccttgccatccatccaccagggctctaccaaggtcgctggaacagaccatgccaccccaccctccctccaactccgctgatcatccactagaaaccagcatgggtagcaagggatggagagaggagacggggaccatggctagaccaggcttgatggtatggatgaaggttgctagatactcacaaggctttaaaaaaaacattcttgcatttatcattaaatacagtgaaaagctcccggatagcaagagcagggggaggctctgtattctgtgtctgtgtgtctggctgtgtctgtgtgtctgcctgtctgggagttcccttgttttttaggccctaagtatcttggtcagtgatatctgccatggctcatataatgtctttttatggataaacatattttcaagccatgattaagtaaagagttttgtgtgagacagaaaccagaaatcagaaatctaggtgaacatacaaattgcacgtgcatgtgtgcacacacagacacacacacagacacacacacagacacacacacacagacatacacagacacacatagacacagacatacacagacacacaaacacacagacacacagacacacacacacacactggggggttggaggaggactgtgtatcatggtacattccattgatctcagcacacaagagttggagacaggaagatcatgagtttgagtttgaaagcagtcgggctgtatagaaattcgaatgccagcctcatagtgagagctgtctcaaagtaaatgagtaaataagatgccaagggatgtagttcatttggtagagtgagagctgagcatgcccacgaagcccggacttgatctcagcaccataagaaaccaggtatagcggcataagcctctaataccagcgctcaggaggtagaagcagggaaatccaaagttcaaggtcaccctgggctacatgagatcttatattaaaaaactaaacacattattttttaaagatttatttatttatttattttatgtgagtgcactatagctgtcttcagacacaccagaagcgggagttggatcccatttcagatggttgtgagccaccatgtggttgctgggaattgaactcaggaattctggaagagcagtcagtgctcttaaccactgagccatctctccagccccaatacattatttaaagaagaagaagaattagaagaagaaggagaaggaggaggaggaggagaagaagaagaagaagaagaagaagaagaagaagaagaagaagaagaaggaggaggaggaggaggaggaggaggaggaggaggaggaggaggaggaggaggaggaggagaaggagaaggagaaggagaaggaaggagaaaaggagaagaagaagaagaagaagaagaagaagaagaagaagaagaagaagaagaagaagaagaagaagaagaagaagaagaagaagaagaaagagttaagagtataaaaccatcttgagaaaagccagatacatcctcattagagtcagctgtgtgagatgttctgtccctgtctttgtttcatttcctgtgggtgatataaaataccccaaccaaagcagcttaagagagaaggggcttctctgacttacaggtccaggatgcagtccacccttgtgtggcagctaaggtggccgaaacccgaagcctttactcatatcacaccctccatcaaaagcagggatcaatgaatttgcatgcacacttatcccagctcacttcctctttctatacagtctaggacccaaatccatttagactgggttttcccacatcgattacagtaataaaaacaatcccctgcaggcatgctcacagatgacctaatctaggtaatccaccattgagattcccttccggtaactttaggttctgttaaattggcaattaacactaaatcatcacacgcctgacgtcaagggtgtgcccctcctgcccgagcaggttctggccagccctgatactttaaatctttaataacagagtcataggtatgtgggtgagaatccagtttcttccattcttttgttgttcatctggcctccttctcattttcctcaagtggagcaagaagcagaaggcattgaggtccacatgagggtaccccgatagttgctgtgctaagagttactcatggccctgacactctacccatttccttaagatgttcttttagagttctgggagataaaacgtgatgtaatatgccttgacagtctaggcccctcagtccttgtgagaccttcttcctgctcaggacgagcatctacctggcatggcattttcgtttggtgaagacaccagactcagaaccagaggacagggctcttcatgagcaacaatagcttagagtttacaatgtgaagcaaggagctgtctaagactgtgagattcccctagacctcccagtagcggggccattagggagactgaaatagaatctcaatatgacccctttggtcaaagaatttgtccaagaaaacaaaacaaaacacttctgaaatcccagccacaaggagtccagcataacaaggtcctgttgcagacacataggtgcctgtgctcacgtttgtacatatatatatatttacatgtgtgtatatatgtgtgtatatatatgaaaaaagtttactttctagaagctaatttcatctaatgctgccctcttgtggccaattggaaatcttacctctgaattctgaaaatttcagtctttgccactttggaacacttaccaggagctaggagggcactggctggctcacattagttacgtttttaggaatttcatgggttttgttttgtttcgtttcttttctttgtttgtttgtttgtttgtttgtttgtttgtttgtttgtttgttttgagacagggtttctttgtgtagccctggctgtcctggaactcactttgtagaccaggctggcctcgaactcagaaatccacctccctctgcctcccgagtgctggaattaaaggcgtgcaccaccatgcccggcctcattttttgttttaaatgttcgccctgggaatttaatccaaggccttgagcatgctagacacatgttctattcagctatgatccagccctttgaactgttttgttaatacttttctctctttttctgtttcgtaatgttgatttttgggtgggattggagcacgagcatccggaagttagaggacaagttttgagagttggttttctccttcattcactaggattggtgccaaactcccttactccctgagacatctttctcccccagtcggattgttaggttttgtgtgtttgatttggtttagagcagtagtccttagccttcctaattctgtgaccctttaatatagttctgtatgtttaatggctaatcttactccaactcagcagttattagtccacaggactaaatgaggaaaaatccacaactgccaagtgtcagacattgcaccgcaggccacagacatgcacaacccagccctgctcgtaggaactagtagactggaagaaaaaaaaaacttgaaatatttttgtggtgctgaggatacaggtcaatagtagaatgttttctcagccctgggttcaatccttagtatagaagaaaaagaagaaagggagatatgggtggaaggaaatacggagggaggaagggaatgagggagggagggagggagggacatttgttgaaagtctaagatggagatatgtaagcagaaggacatttaatctacatgggaatcaaaagacagctgactgagctggtatactctgaagtgtcagataaaacgagccaggaaacatctcctggaagaggaagctggggcacctatggaattgtgaatctgtgtggtgatgtctgagagaaggttgagttgtaggacataaaatgaatgggcaccatggccacaatgataccacactgcccagacagtcagaaatactgtcatggaagatgaggccgcattgaggagctgggccaagatgctgatggtggtggcacctcagatcacctcacctaggagagaaaccgttatcttggccttctgggtgatctgctgcgtagaggcttaccgtgggcaatgtgttgacaatatgattgcattatggtggaataaatccacatagaagttggtgactgtgcttttgtgggtctgtggatctgagtgaatggaggaaacaaaccaaggggtatagaaaagaattgagcaaattagtatgactggtaagctggataaaaagatggagtatggaagaaggatgagtcagagaggggccgtgggtttctcagacagggagcctctgtagccactacatc

SEQ ID NO: 7 - DNA, 6574 bp, Artificial Sequence
  synthetic nucleic acid sequence
gacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctggtaagtgtcccttctgtgactaccaagaacaaaagaactgcagcaaagagtgagacagagcccagcttttgttcactcagccctatagacacaaatgggccagtccagtctacaggataactaattttagtggttcctgaaaagtcattcactaaacacaattttttttttttttggagacagactctcactctgttgcctaggctggagtgcaatggcacgcgatctcggctcactgcaacttccgcctcctggcttcaagtgattctcctgcctcagcctcctgagtagctgggattacaggcaccctccaccatgcctggctaatttttgcatttttagtagagatggggtttcaccatgttggccaggctggtctcaaactcctgaccttgagtaatctgcccatctcagccacccaaaatgctgggattacaggcatgagtcacagtgcccggccacacaattttatttttatttttgagacagtctcgctctgttgcccaggctggggtgcggtggtacaatctcagatcactgcaacctccacctcctgggttcaagtgaatcttgtgccacagctgcccaagtagctgggactacaggtgtgcaccaccatgcctggctaatttttttatttttagcaaagatggggtttcgccatgttggccaggctggtctcgaactcctggcctcaagtgattctcctgcctgggccttataggcgcacaccaccatgcctggctaatattttgtattttttgttagtgatggggtttccccatgttgtccaggctggtcttaaactcctgggctcaagcagtcctcctgcctcagcctcccaatgtgctggaattataggtgtgagccaccacacctgacctagacaccctcttttatcctgagctggaatactgatcacccccctcactggggcactcagactcaactcctttgccaaactcttcacaatagagagggctgttctagaagtttccttgtcattgtgttaagatggcgtctctaacttcaagcattctgaacagccttagggagcaaaggatcagtgtgttgtggagtgagaccactcctgctgctgtcaaacaacacagagacaattgacagcaagcaaggagtcccttttggtgagagacacacacatgcacacataacatacatgacatacacagacacgtaacatacatggcatacacagacacgtaacatacatgacatgcacagacacgtatacacacgctcaaaaacatgtcgtgggcattgccatatgcacttgaatacccagaggccggtgctgggagggcatactctggtttcgtatttgtaagctcagcagccagacaccaccagacacatcgtgggccctggacaggtgtgcttctcaagtgaatgaatacatgaacagtgttttctatgaagcatttgtgaaattatctgcatcattccatctatattttctagccactcggaacacaacgaaacaagtgacacctcaacctgaagaacagaaagaaaggaaaaccacagaaatgcaaagtccaatgcagccagtggaccaagcgagccttccaggtgagagatgaatctgtcctccagctaactcctgctagcgcacccttctcctgcaggcagatgatgtgtacccaaaggaagaaggtctgccctgccttctataaacacatgcatgtggctccgtcctcccaagacatcattgcagacaagaattagtgcagagttgctcagaggctgtttgctcagcaactaaagccaatctgacggcagccaatccaggtctgggcttactccctggtttggtaactggccaaatagaattcaacccaaattcaactcaacttaaaagcgtctgttgggtctgtgtgccaggccctgctgatagaagacacaaacaagagtgtgacaacgatccctgccctcaaggaaacgctctgcgtcttccccttaagcacaagacaacgcaccgcaaaaaaattacttgcttcccagtatttcttccagggcaaggttgtaaaatgtgcatgccacacttgtgagtctcaaaagctccagatgcgtgtgtcctggccagcagtctgtgcatgagtgggcactgtggctgtcccttgtctgccctctgctttcccacgactgaattgggattcagttatggtgtcaagaaaatgggctgggcgcagtggctcacgcctgtaatcccagcactgtgggagtctgaggcaggcagatcacttgaggtcaggagttcgagaccagcctggccaacatggtgaaaccttgtctctacaatcatacaaaaattaagctgggcatggtggtggatgcctgtagtcccagctactcaggaggccaaggcatgagaattgcttgaaatcaggaggtggaggttgcagtgagcagagattgcatcactgcactccagcctgagtgacagaggaagactctgtcccccctcaaaaaaacaaaacaaaaaagaaaaaaaacccacagaacctgctaggaagaattgagctttctaaggtcaggctagccttaggagacttcagagccctttgctgattttacaaccttacaaagaactgtatcagataccatcccatttgagcctggctatacaatgagtcaggaccaggaagggttacttcaacttactgaagaaactgagatgcacaaaagataaaaagatttggttaagctcacatagtagggctggaatcagacctcaggtctcccagattagatgaggaaactttctgctgattacttattttaaaagagtttgaatttacttacagcttccattattttattttttaaaacagactaatctaggggcacagttgcaacatgatgctcttactccttccccttccgactttgtcctggaatggtgtttccaagcacatttgtttgttttgttctacttcccccatatcaccctgcacagttattggaaggcttcaaagaggccaatgcttgttgtcctttctcgaaggctggctctgcctgagggatgtttcaaagattgtctcagtttgggtgttttcattggaaagaagcaaacccaattcaaacatgttcagacagttacgaattgtactggctgtcaaacaaccttttatcctgagaccaaaacacaagttcagtcgtgatgttcaaggtgccacatggcaggagccagcctgggacagggccacccagcttcctgctggggaagaggctctggaggatatttacagaggctctgttgggtatgcagggttggggagagtctggaagttcagagcacgtgcgcagtttcacaggctggggttagagggcacatacaattccatatgtccaggttgggggctgtgggtggggaattcagagatttaatcagaacaagacatggtctcaggccacaggtccgtcaggcctgggatgtagttgggtgagtggaaagttctggggatgggaaattttgagacgtcttgaaagggcgcatgtgtgtcaaccttcaggtgtggcttgatcagagatggtgctccatttctctgaggttccctcagcccgtccctcctcctggctttggtgttgtccccaatccttccgacatcatcccaagatgcccagccgctgcccccaagaatgcacttcctcacttgtccagcagaaatccaaggtggtcttcatgctccattggcccaaataggcttgagcctacccttgtgtctcagctgagactgccccgcccactcctgtccactgtcaacacctgcactgtcttccaagaggcagttgtggcgttcctgtctcggggccgcaccaaccagaatgaatcgctcattcctctgtgttcccattgccaacgtctgctatccttcacatgcctttcttacccaatccactgtccaccccttgcaggagtgacagggtcttcgtgccttcctacagggcctagcaaacttccccacgctcagacgttctcaacatatattggttgaatgaaaggatgagcaagcagtcaagagagtaagaaccaaggtccagtccctagcaagaggcaacctggactcactcgcggtgctgtccccaacttgccttcagagcattccttccatcttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtagggtggctcccttctggggtccacaatgttgctttgctccatcaggctgaccagagtggaatccctgaccactcctgctggacaagagtggtccttggacaagacccttgatctccctgggcttccattttttctcattctgagaaaaaaaaaaaaatttaaatagaccagatcagatggactctaagggctccttcaatgaaggtccccctgtgccctgagtggggagaaaggttggagacagaagagaaggaaagccctctcctaagcaccctagatgctgcctcgtagttggtcctaagtccactacttctctaagccaccaaggcctcctctggctccaagggcaagtccagtttcccactcacacatttttcctcttactggtgatcgtgtaaatgcatgatctgaacagccctccacggtcagcatgttccttccccttgtgccattccccagctgagcttgtaaaaataaacataaaatgtgcagagtcgggacttactcagtgcacatgcgcgcacacacacacacacacacacatactcatgcaaatgctcagcccaccgatgtcgaggtctcctgctgtcttccttctgaaatcttattagtatcacgacaatcaaaagtgacttacatgcaggctgggcacagtggctcacacctgtaatcccagcactctgggaggccaaggcgggcagatcactttaggtcaggagttcgagaccagcctggccaacatggtgaaaccccgtctctactaaaaatacaaaaattatctgggcgtggtggtgggtgcctgtaatcccagctacttgggaggctgaggcaggagaatcacttgaacccaggaggtggagttgcagtgagctgagatcacaccactgcactccagcctgggcaacagcaagactccgtctcaaaaaaaaaaagtgacttacattcaaccacaagtattcctggttttcttgctcactgcacttctcatactccatgtccttctcttgggttccctagggaggctgttttgttcccttagctgcttccctgggccaccctgacagccagggaatgcttgctgcttccttggaagagggtgagaggcagggcagagccagggttgctgacccctcaggagaagtggagagcctggcacagaagcagctgtcccttagtgcatctctgctccatccagtggttcaatggcattcgtattattatgacctgtttccaagtcacagatggaaaccaggactggggagggaggctggggagaaagcattgctcagagttttggggacagctagggtcagagttagaattcagcctcgtctgctctgctgagaccacttcaccgcttcccagtgcccctgctgtggaaccatgaggggtggcatccctggcgaagtgcccacaccaggattcctgcctcctcgggcgtgtctggggccggctgcctggggcaggcggggtgggagtgaggccctgactcctgtgtttagctccaccagcatcacttactctctcccccaggtgaagagaagcctcaggcaagccccgaaggccgtcctgagagtgagacttcctgcctcgtcacaacaacaggtgcgggagaagacaaacgctggaccacagaggcctagtccaaaagggcagaggtgaccaggagccaggctcagggagataggcggaggtgacctgtaggggagaagcccacagcagcctcctctccctctgagcagggacagggcctcagcaacctgcaggccccaaagcaagtgtcagaaagagggaacccaggaccaggaccaagcacggtccccaggcagagatggaacaccttccctcaccaccaccacgtgtctcccaccagcctctgagcttctcattcacagagacaccctgacttcctttagcctcgtgctgtcctaaagtcacggtagcaggagtgtctctctttatctctttttcacagattttcaaatacagacagaaatggctgcaaccatggagacgtccatatttacaaca

SEQ ID NO: 8 - DNA, 50 bp, Artificial Sequence
  synthetic nucleic acid sequence
tctccttgcctttgcagaactgtgtgacgatgacccgccagagatcccac

SEQ ID NO: 9 - DNA, 50 bp, Artificial Sequence
  synthetic nucleic acid sequence
catggagacgtccatatttacaacagagtataaggtagcaggtgggccag

SEQ ID NO: 10 - DNA, 4440 bp, Artificial Sequence
  synthetic sequence
aagttcctgctgagcagatcagcctaatgcttaaatagaacaactcctggctgtcattgacattgtctaaaagccaagatgacagactgagaggcctgagcccttgttctggcattctcccaggaagatgcagtaaaggggttgacccaatatactgcagagaatttcatccagttccctcctccatcctgatcccatgtgccaggaagatggagccacgcttgctgatgttggggtttctctcattaaccatagtacccagttgtcgggcagaactgtgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtataaggtagcagtggccagctgcctcttcctgctcatcagcatcctcctcctgagcgggctcacctggcaacacagatggaggaagagcagaagaaccatctagcaagctagaaaagtcagagcccaggcaagcggatgggaatcacaaagctcaagccaaatctgagacgccaagcattcacctaacggctgtttccttctgatccctgggtttctagaacattctgaagtcacaggacataacagcaactctatcactaaactggactttgccattgaagaataggatctaaccacttcagcacagcagttctaaagctttaatgggagagagggcccaacagtgctctgtgtgttttgtttttgtgtatatctgttgatgggagctgagatggtgtggtcacttttcatgtaacatatagtatagaaaaagtagctttaggttgacttcattgttacaacccagtttggaaagcccaagtaaaactcagcactaatgtaaataattcctcctcctcctcctctctcttttcatcctccgctccatcttcctcttcttcctcctccttttccacctcctctgtccctacccacccccacccatccactttccttcttcctttctgctctcacaagctcatcctagctacacgtgcatggctggctcctttttcaacctctgtttgcctaactggctcttctgatttcatcacttactgatcagcctttaaaactctgagctggcaaagatgactctatctatgttcttggctcagtcccagaaggaaacccccttttcatgaagcttcagttttgacatcctgaagaacagaaactgtggcagaacaatcttcagataacatcaaaacaaagtggagaagccacgggaactgtggagctctggtattcagaagcctgtgtctagggtctgcgccaggagcagaaggctgaaggaagtcccaggacgtggacttagatgctttcccagcaggccactctaagcgctggtttctttgggacagctgtcaattgtacgctcaatttagcctgcactaatctgatgcttacaggtgaacactcaaggcacaggtatggacttggtacataccgtgaaaacactggaaagaaaagaatactttcaagtttacagaaggaaggaaggaaaaaggaagcagaggtggtgattatacaaaagattagctgtagactggatatcccaggcatcctcggataatgcccccgccccagcaccctgatccaggtcaccaaagccttgtgagatcagactgcagagccagtctgtctctgagtcagtaaatgtagaatttggatttctcacaagttcctggcggtgtcttttttttttttaatattttttattaggtattttcctcatttacatttccaatgctatcccaaaagtcccccataccctcccccacactcccctacccacccactcccactttttggccctggtgttcccctgtactggggcatataaagtttgcaagtcctggtggtgtctttatgctgatctctagcccacactttgtgaggcactgggctatcccagtgtgctctcctcttccacagataccaaaagcacctgggtttgatgctcagacttctgagcacgttcttgttcaatctcttgcgtaagatttcctctcagatgagttgagtcagattctcatgtttaacagtgttttaggggattcacagaagcccaaactatcagttttcatttctgaaaaggctggaaaattttatgaaaaactttcaaaggtcagacagagccattttgagtcttttatgtgaccaagtatgaaccagatctttcctatctatggtctcccctttccaaaatatatcttttgtggggacacggcaaggaggaaagttaaatagaatctcaagctactaattttagaaaagaaaaaaatattaaactcttactaaagagctgtgggtagtggtacacacctgtaagcccagctctcaggagactgaggcaggaggattgcagtgagtcagagatcagcttcacctacaagcaaaaccctgccttacccctcaacctttccataaaaacagtcttacttgtgtaaaattaatttttaatacatatttgtgcacgatgtgtgtgcctggtgcccagggaagccaaaaaaatatgttcaatgtccctggaataggtggttatgagctgccatggaggtgatgggatttaaacccctgttctctgaaaaagcagctagttctcttaaccactgagccatctctacagccccattaaattgaattttattgtcattactcaatatgggagatggggtaatgataacaattttttttttataatactaagatgtttagctattttactctccttcacaagtgtagagtagaattttctagaagctacatgcatgatattatcgctctggtgttaacacataatggatttatcttgttaataaaagaattagtaaataatattttaaattttttctttgttttagttttaagatgattaatatctatagatactagtgtacattaagaaagcctttgggatcctcaatcattttgcatggttttagtaattttttaataacataaagaaggtctgacagattatgctaaagagctattgtggtatggattagaaatggcccccacaggctcctgtgttcaaacatcagctagcagtgctgctttgggttttggaacctttaagaggtggggccttgcttaaggagataggtcactggaggtgaaccagacttgcttccagtctccctctctgctgtcccaggaagccatcatatgaggagtttcacaacatacttctgctgccacagagtccctccagtaaggcacgtactggccactgtgtagtgtaccctctaaaactgagctagagctcccctctcctccctacactatctctgttgatgctttgtcacggtgatgaagaacataagtaaggcaaagacaagacatagtttggagactcacgtgagcatctcagccagactcaggcacagctgcgatgtgggaattatcaagcatgagatgcaaagcaatggaaaatgaatcgttatgacagaagcctacatctagtcttcccttcttcccattagtaataatagcccgtgttttagaagaacacatctttttggtgttctaggtagcttatattgcaaatgtggcacaatctaagagaaatctgggatgagggaacctcagtgaaagattctccttgatcagattgtcccactgatgattgatatgggaaggcccaacccactgtgaaaggcaccacccattggcaggatgactggggttatagaagcaaacaggctgagcatgagccagtgagcaagccagtaagcagtggcttctctttggaagaagcatggctttcttccgtggcttctgtttgatctctctcaatgatagattgtgacctagaagtataagctaaaataaaccatttcttacccata

SEQ ID NO: 11 - protein, 272 aa, Artificial Sequence
  synthetic sequence
MEPRLLMLGFLSLTIVPSCRAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYKVAVASCLFLLISILLLSGLTWQHRWRKSRRTI

SEQ ID NO: 12 - DNA, 80 bp, Artificial Sequence
  synthetic sequence
ggcagaggccagcctggtctgcatagtgaattccagtatagtcgacggtatcgataagcttgatatcgaattccgaagtt

SEQ ID NO: 13 - DNA, 80 bp, Artificial Sequence
  synthetic sequence
ttcatcagtcaggtacataatggtggatccactagttctagagcggccgcgccagaacgacatagtgagaccttgtttca

SEQ ID NO: 14 - DNA, 25 bp, Artificial Sequence
  synthetic primer
tctggtggaatttttggggttcact

SEQ ID NO: 15 - DNA, 26 bp, Artificial Sequence
  synthetic primer
tctgtctcactctttgctgcagttct

SEQ ID NO: 16 - DNA, 20 bp, Artificial Sequence
  synthetic primer
gctcgactagagcttgcgga

SEQ ID NO: 17 - DNA, 25 bp, Artificial Sequence
  synthetic primer
ctgagtgggtctgagcaagatgtcc

SEQ ID NO: 18 - DNA, 25 bp, Artificial Sequence
  Synthetic nucleic acid sequence
gatattccggtgtgccattctgcct

SEQ ID NO: 19 - DNA, 25 bp, Artificial Sequence
  Synthetic nucleic acid sequence
gtagtaggcagcgtcaactcgaagg

SEQ ID NO: 20 - DNA, 26 bp, Artificial Sequence
  Synthetic nucleic acid sequence
gaagagcagaagaaccatctagcaag

SEQ ID NO: 21 - DNA, 25 bp, Artificial Sequence
  Synthetic nucleic acid sequence
cattagtgctgagttttacttgggc

SEQ ID NO: 22 - DNA, 23 bp, Artificial Sequence
  synthetic oligonucleotide
ggatcggccattgaacaagatgg

SEQ ID NO: 23 - DNA, 23 bp, Artificial Sequence
  synthetic oligonucleotide
cagaagaactcgtcaagaaggcg

SEQ ID NO: 24 - DNA, 25 bp, Artificial Sequence
  synthetic primer
actgatcccgaggaggtagagtacg

SEQ ID NO: 25 - DNA, 25 bp, Artificial Sequence
  synthetic primer
agggtcacacttacagttgctggtg

SEQ ID NO: 26 - DNA, 25 bp, Artificial Sequence
  synthetic primer
ctgggctctgtctcactctttgctg

SEQ ID NO: 27 - DNA, 25 bp, Artificial Sequence
  synthetic primer
catgggtcagctgtagtacgttggg

SEQ ID NO: 28 - DNA, 25 bp, Artificial Sequence
  synthetic primer
aggctaaatgggtagggagactgct

SEQ ID NO: 29 - DNA, 25 bp, Artificial Sequence
  synthetic primer
gacaagcgttagtaggcacatatac

SEQ ID NO: 30 - DNA, 24 bp, Artificial Sequence
  synthetic primer
gctccaatttcccacaacattagt

(19) United States
(12) Patent Application Publication
(10) Pub. No.: US 2022/0312749 A1
(43) Pub. Date: Oct. 6, 2022

(54) GENETICALLY MODIFIED NON-HUMAN ANIMAL WITH HUMAN OR CHIMERIC IL2RA

(71) Applicant: Biocytogen Pharmaceuticals (Beijing) Co., Ltd., Beijing (CN)

(72) Inventors: Yuelei Shen, Beijing (CN); Yang Bai, Beijing (CN); Chengzhang Shang, Beijing (CN); Rui Huang, Beijing (CN); Meiling Zhang, Beijing (CN); Jiawei Yao, Beijing (CN); Chaoshe Guo, Beijing (CN); Yanan Guo, Beijing (CN)

(21) Appl. No.: 17/633,668

(22) PCT Filed: Aug. 7, 2020
(86) PCT No.: PCT/CN2020/107886

§ 371 Date: Feb. 8, 2022

(30) Foreign Application Priority Data
Aug. 9, 2019 (CN) 201910734610.7
Jan. 6, 2020 (CN) 202010011394.6

(51) Int. Cl. A01K 67/027; C12N 15/85; C07K 14/715; A61K 49/00

(52) U.S. Cl. CPC A01K 67/0278; C12N 15/8509; C07K 14/7155; A61K 49/0008; C12N 2800/107; A01K 2207/15; A01K 2217/072; A01K 2227/105; A01K 2267/03

(57) ABSTRACT

The present disclosure relates to genetically modified non-human animals that express a human or chimeric (e.g., humanized) IL2RA, and methods of use thereof.

### CLAIM OF PRIORITY

This application claims the benefit of Chinese Patent Application App. No. 201910734610.7, filed on Aug. 9, 2019, and Chinese Patent Application App. No. 202010011394.6, filed on Jan. 6, 2020. The entire contents of the foregoing are incorporated herein by reference.

### TECHNICAL FIELD

This disclosure relates to genetically modified animal expressing human or chimeric (e.g., humanized) IL2RA, and methods of use thereof.

### BACKGROUND

The immune system has developed multiple mechanisms to prevent deleterious activation of immune cells. One such mechanism is the intricate balance between positive and negative costimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

This disclosure is related to an animal model with human IL2RA or chimeric IL2RA. The animal model can express human IL2RA or chimeric IL2RA (e.g., humanized IL2RA) protein in its body. It can be used in the studies on the function of IL2RA gene, and can be used in the screening and evaluation of anti-human IL2RA antibodies. In addition, the animal models prepared by the methods described herein can be used in drug screening, pharmacodynamics studies, treatments for immune-related diseases (e.g., autoimmune disease), and cancer therapy for human IL2RA target sites; they can also be used to facilitate the development and design of new drugs, and save time and cost. In summary, this disclosure provides a powerful tool for studying the function of IL2RA protein and a platform for screening cancer drugs.

In one aspect, the disclosure relates to genetically-modified, non-human animals whose genome comprises at least one chromosome comprising a sequence encoding a human or chimeric IL2RA. In some embodiments, the sequence encoding the human or chimeric IL2RA is operably linked to an endogenous regulatory element at the endogenous IL2RA gene locus in the at least one chromosome. In some embodiments, the sequence encoding a human or chimeric IL2RA comprises a sequence encoding an amino acid sequence that is at least 50%, 55%, 65%, 70%, 75%, 80%, 85%, 90%, 95%, 99%, or 100% identical to human IL2RA (NP_000408.1 (SEQ ID NO: 4)). In some embodiments, the sequence encoding a human or chimeric IL2RA comprises a sequence encoding an amino acid sequence that is at least 50%, 55%, 65%, 70%, 75%, 80%, 85%, 90%, 95%, 99%, or 100% identical to SEQ ID NO: 11. In some embodiments, the sequence encoding a human or chimeric IL2RA comprises a sequence encoding an amino acid sequence that corresponds to amino acids 25-237 of SEQ ID NO: 4.

In some embodiments, the animal is a mammal, e.g., a monkey, a rodent or a mouse. In some embodiments, the animal is a mouse. In some embodiments, the animal does not express endogenous IL2RA. In some embodiments, the animal has one or more cells expressing human or chimeric IL2RA. In some embodiments, the expressed human or chimeric IL2RA can bind to a human IL2. In some embodiments, the expressed human or chimeric IL2RA can bind to an endogenous IL2.

In one aspect, the disclosure relates to genetically-modified, non-human animals, wherein the genome of the animals comprises a replacement, at an endogenous IL2RA gene locus, of a sequence encoding a region of endogenous IL2RA with a sequence encoding a corresponding region of human IL2RA. In some embodiments, the sequence encoding the corresponding region of human IL2RA is operably linked to an endogenous regulatory element at the endogenous IL2RA locus, and one or more cells of the animal expresses a chimeric IL2RA. In some embodiments, the animal does not express endogenous IL2RA. In some embodiments, the locus of endogenous IL2RA is the extracellular region of IL2RA. In some embodiments, the animal has one or more cells expressing a chimeric IL2RA having an extracellular region, a transmembrane region, and a cytoplasmic region, wherein the extracellular region comprises a sequence that is at least 50%, 60%, 70%, 80%, 90%, 95%, or 99% identical to the extracellular region of human IL2RA. In some embodiments, the extracellular region of the chimeric IL2RA has a sequence that has at least 10, 20, 30, 40, 50, 60, 70, 80, 90, 100, 110, 120, 130, 140, 150, 160, 170, or 180 contiguous amino acids that are identical to a contiguous sequence present in the extracellular region of human IL2RA. In some embodiments, the animal is a mouse, and the sequence encoding the region of endogenous IL2RA is exon 2, exon 3, exon 4, exon 5, and/or exon 6 of the endogenous mouse IL2RA gene. In some embodiments, the animal is heterozygous with respect to the replacement at the endogenous IL2RA gene locus. In some embodiments, the animal is homozygous with respect to the replacement at the endogenous IL2RA gene locus.

In one aspect, the disclosure relates to methods for making a genetically-modified, non-human animal. The methods involve replacing in at least one cell of the animal, at an endogenous IL2RA gene locus, a sequence encoding a region of an endogenous IL2RA with a sequence encoding a corresponding region of human IL2RA. In some embodiments, the sequence encoding the corresponding region of human IL2RA comprises exon 1, exon 2, exon 3, exon 4, exon 5, exon 6, exon 7, and/or exon 8 of a human IL2RA gene. In some embodiments, the sequence encoding the corresponding region of IL2RA comprises exon 2, exon 3, exon 4, exon 5, and/or exon 6 (or part thereof, e.g., part of exon 2 and/or exon 6) of a human IL2RA gene. In some embodiments, the sequence encoding the corresponding region of human IL2RA encodes amino acids 25-237 of SEQ ID NO: 4. In some embodiments, the region is located within the extracellular region of IL2RA. In some embodiments, the animal is a mouse, and the sequence encoding the region of the endogenous IL2RA locus is exon 2, exon 3, exon 4, exon 5, and/or exon 6 of mouse IL2RA gene (e.g., part of exon 2, exon 3, exon 4, exon 5, and part of exon 6).

In one aspect, the disclosure relates to non-human animals comprising at least one cell comprising a nucleotide sequence encoding a chimeric IL2RA polypeptide, wherein the chimeric IL2RA polypeptide comprises at least 50 contiguous amino acid residues that are identical to the corresponding contiguous amino acid sequence of a human IL2RA, wherein the animal expresses the chimeric IL2RA. In some embodiments, the chimeric IL2RA polypeptide has at least 50 contiguous amino acid residues that are identical to the corresponding contiguous amino acid sequence of a human IL2RA extracellular region. In some embodiments, the chimeric IL2RA polypeptide comprises a sequence that is at least 90%, 95%, or 99% identical to amino acids 25-237 of SEQ ID NO: 4. In some embodiments, the nucleotide sequence is operably linked to an endogenous IL2RA regulatory element of the animal. In some embodiments, the chimeric IL2RA polypeptide comprises an endogenous IL2RA transmembrane region and/or an endogenous IL2RA cytoplasmic region. In some embodiments, the nucleotide sequence is integrated to an endogenous IL2RA gene locus of the animal. In some embodiments, the chimeric IL2RA has at least one mouse IL2RA activity (e.g., interacting with mouse IL2, and promoting immune responses in mice) and/or at least one human IL2RA activity (e.g., interacting with human IL2, and promoting immune responses in human).

In one aspect, the disclosure relates to methods of making a genetically-modified mouse cell that expresses a chimeric IL2RA, the method including: replacing, at an endogenous mouse IL2RA gene locus, a nucleotide sequence encoding a region of mouse IL2RA with a nucleotide sequence encoding a corresponding region of human IL2RA, thereby generating a genetically-modified mouse cell that includes a nucleotide sequence that encodes the chimeric IL2RA, wherein the mouse cell expresses the chimeric IL2RA. In some embodiments, the chimeric IL2RA comprises a signal peptide sequence (e.g., a mouse signal peptide sequence or a human signal peptide sequence), an extracellular region of mouse IL2RA, an extracellular region of human IL2RA, a transmembrane and/or a cytoplasmic region of a mouse IL2RA. In some embodiments, the chimeric IL2RA comprises an extracellular region of human IL2RA comprising a human signal peptide sequence; and a transmembrane and/or cytoplasmic region of mouse IL2RA. In some embodiments, the nucleotide sequence encoding the chimeric IL2RA is operably linked to an endogenous IL2RA regulatory region, e.g., promoter.

In some embodiments, the animals further comprise a sequence encoding an additional human or chimeric protein. In some embodiments, the additional human or chimeric protein is programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), Glucocorticoid-Induced TNFR-Related Protein (GITR), T-cell immunoglobulin and mucin-domain containing-3 (TIM-3), Signal regulatory protein α (SIRPα) or TNF Receptor Superfamily Member 4 (TNFRSF4 or OX40).

In one aspect, the disclosure relates to methods of determining effectiveness of an anti-IL2RA antibody for the treatment of cancer, including: administering the anti-IL2RA antibody to the animal as described herein, wherein the animal has a tumor, and determining the inhibitory effects of the anti-IL2RA antibody to the tumor. In some embodiments, the animal has one or more cells (e.g., T cells) that express IL2RA. In some embodiments, the animal has one or more tumor cells that express IL2RA.

In some embodiments, the tumor comprises one or more cancer cells that are injected into the animal. In some embodiments, determining the inhibitory effects of the anti-IL2RA antibody to the tumor involves measuring the tumor volume in the animal. In some embodiments, the tumor cells are melanoma cells, leukemias, lymphomas, solid tumor cells, colorectal cancer cells, ovarian cancer cells, prostate cancer cells, melanoma cells, lung cancer cells, breast cancer cells, gastric cancer cells, esophageal squamous cell carcinoma (ESCC) cells, and/or head-and-neck cancer cells.

In one aspect, the disclosure relates to methods of determining effectiveness of an anti-IL2RA antibody for the treatment of various immune-related disorders, e.g., autoimmune diseases.

In one aspect, the disclosure relates to methods of determining effectiveness of an anti-IL2RA antibody and an additional therapeutic agent for the treatment of a tumor, including administering the anti-IL2RA antibody and the additional therapeutic agent to the animal as described herein, wherein the animal has a tumor, and determining the inhibitory effects on the tumor. In some embodiments, the animal or mouse further comprises a sequence encoding an additional human or chimeric protein. In some embodiments, the additional human or chimeric protein is PD-1, CTLA-4, LAG-3, BTLA, PD-L1, CD27, CD28, CD47, CD137, CD154, TIGIT, TIM-3, GITR, SIRPa or OX40. In some embodiments, the animal further comprises s sequence encoding a human or chimeric programmed cell death protein 1 (PD-1). In some embodiments, the animal further comprises a sequence encoding a human or chimeric programmed death-ligand 1 (PD-L1). In some embodiments, the additional therapeutic agent is an anti-PD-1 antibody or an anti-PD-L1 antibody.

In some embodiments, the additional therapeutic agent is an antibody (e.g., human antibody) the specifically binds to IL2, IL2RB, IL2RG, PD-1, CTLA-4, LAG-3, BTLA, PD-L1, CD27, CD28, CD47, CD137, CD154, TIGIT, TIM-3, GITR, SIRPα, OX40, CD20, EGFR, or CD319. In some embodiments, the additional therapeutic agent is an anti-PD-1 antibody (e.g., nivolumab), an anti-PD-L1 antibody, an anti-CTLA4 antibody (e.g., ipilimumab), an anti-CD20 antibody (e.g., rituximab), an anti-EGFR antibody (e.g., cetuximab), or an anti-CD319 antibody (e.g., elotuzumab). In some embodiments, the additional therapeutic agent is an anti-PD-1 antibody or an anti-PD-L1 antibody. In some embodiments, the tumor comprises one or more tumor cells that express IL2RA, PD-1 or PD-L1.

In some embodiments, the animal comprises one or more cells (e.g., T cells) that express IL2RA. In some embodiments, the tumor comprises one or more tumor cells that express IL2RA. In some embodiments, the tumor comprises one or more tumor cells that express IL2RB or IL2RG. In some embodiments, the tumor is caused by injection of one or more cancer cells into the animal. In some embodiments, determining the inhibitory effects of the treatment involves measuring the tumor volume in the animal. In some embodiments, the animal has acute lymphoblastic leukemia (ALL), B-cell chronic lymphocytic leukemia (B-CLL), hairy cell leukemia (HCL), solid tumors, colorectal cancer, ovarian cancer, prostate cancer, melanoma, lung cancer, breast cancer, gastric cancer, esophageal squamous cell carcinoma (ESCC), leukemia, lymphoma, multiple myeloma, sarcoma, and/or head-and-neck cancer.

In one aspect, the disclosure relates to a method of determining effectiveness of an anti-IL2RA antibody for treating an autoimmune disorder, comprising a) administering the anti-IL2RA antibody to the animal as described herein, wherein the animal has the autoimmune disorder; and b) determining effects of the anti-IL2RA antibody for treating the auto-immune disease. In some embodiments, the autoimmune disorder is multiple sclerosis, rheumatoid arthritis, juvenile idiopathic arthritis, type 1 diabetes, aplastic anemia, asthma, idiopathic thrombocytopenia, uveitis, and eczema.

In one aspect, the disclosure relates to a method of determining effectiveness of an anti-IL2RA antibody for inhibiting immune response, comprising a) administering the anti-IL2RA antibody to the animal as described herein; and b) determining effects of the anti-IL2RA antibody in immune response. In some embodiments, the antibody can be used to treat graft-versus-host disease or inhibit transplant rejection (e.g., organ transplantation rejection).

In one aspect, the disclosure relates to proteins comprising an amino acid sequence, wherein the amino acid sequence is one of the following: (a) an amino acid sequence set forth in SEQ ID NO: 11; (b) an amino acid sequence that is at least 90% identical to SEQ ID NO: 11; (c) an amino acid sequence that is at least 91%, 92%, 93%, 94%, 95%, 96%, 97%, 98%, or 99% identical to SEQ ID NO: 11; (d) an amino acid sequence that is different from the amino acid sequence set forth in SEQ ID NO: 11 by no more than 10, 9, 8, 7, 6, 5, 4, 3, 2 or 1 amino acid; and (e) an amino acid sequence that comprises a substitution, a deletion and/or insertion of one, two, three, four, five or more amino acids to the amino acid sequence set forth in SEQ ID NO: 11. In some embodiments, provided herein are cells comprising the proteins disclosed herein. In some embodiments, provided herein are animals having the proteins disclosed herein.

In one aspect, the disclosure relates to nucleic acids comprising a nucleotide sequence, wherein the nucleotide sequence is one of the following: (a) a sequence that encodes the protein as described herein; (b) SEQ ID NOS: 8-10 or 12-13; (c) a sequence that is at least 90% identical to SEQ ID NOS: 8-10 or 12-13; (e) a sequence that is at least 91%, 92%, 93%, 94%, 95%, 96%, 97%, 98%, or 99% identical to SEQ ID NOS: 8-10 or 12-13; and (f) a sequence that is at least 91%, 92%, 93%, 94%, 95%, 96%, 97%, 98%, or 99% identical to SEQ ID NOS: 8-10 or 12-13. In some embodiments, provided herein are cells comprising the nucleic acids disclosed herein. In some embodiments, provided herein are animals having the nucleic acids disclosed herein.

In another aspect, the disclosure also provides a genetically-modified, non-human animal whose genome comprise a disruption in the animal's endogenous IL2RA gene, wherein the disruption of the endogenous IL2RA gene comprises deletion of exon 1, exon 2, exon 3, exon 4, exon 5, exon 6, exon 7, and/or exon 8, or part thereof of the endogenous IL2RA gene.

In some embodiments, the disruption of the endogenous IL2RA gene comprises deletion of one or more exons or part of exons selected from the group consisting of exon 1, exon 2, exon 3, exon 4, exon 5, exon 6, exon 7, and exon 8 of the endogenous IL2RA gene.

In some embodiments, the disruption of the endogenous IL2RA gene further comprises deletion of one or more introns or part of introns selected from the group consisting of intron 1, intron 2, intron 3, intron 4, intron 5, intron 6, and intron 7 of the endogenous IL2RA gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 650, or more nucleotides.

In some embodiments, the disruption of the endogenous IL2RA gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, and/or exon 8 (e.g., deletion of at least 300 nucleotides of a nucleotide from exon 2 to exon 6).

In some embodiments, the mice described in the present disclosure can be mated with the mice containing other human or chimeric genes (e.g., chimeric SIRPα, chimeric PD-1, chimeric PD-L1, chimeric CTLA-4, or other immunomodulatory factors), so as to obtain a mouse expressing two or more human or chimeric proteins. The mice can also, e.g., be used for screening antibodies in the case of a combined use of drugs, as well as evaluating the efficacy of the combination therapy.

In another aspect, the disclosure further provides methods of determining toxicity of an agent (e.g., a IL2RA antagonist or agonist). The methods involve administering the agent to the animal as described herein; and determining weight change of the animal. In some embodiments, the method further involve performing a blood test (e.g., determining red blood cell count).

In one aspect, the disclosure relates to a targeting vector, including a) a DNA fragment homologous to the 5′ end of a region to be altered (5′ arm), which is selected from the IL2RA gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL2RA gene genomic DNAs in the length of 100 to 10,000 nucleotides.

In some embodiments, the DNA fragment homologous to the 5′ end of a region to be altered (5′ arm/receptor) is selected from the nucleotide sequences that have at least 90% homology to the NCBI accession number NC_000068.7. In some embodiments, the DNA fragment homologous to the 3′ end of the region to be altered (3′ arm/receptor) is selected from the nucleotide sequences that have at least 90% homology to the NCBI accession number NC_000068.7.

In some embodiments, a) the DNA fragment homologous to the 5′ end of a region to be altered (5′ arm/receptor) is selected from the nucleotides from the position 11670417 to the position 11674302 of the NCBI accession number NC_000068.7; c) the DNA fragment homologous to the 3′ end of the region to be altered (3′ arm/receptor) is selected from the nucleotides from the position 11683554 to the position 11689329 of the NCBI accession number NC_000068.7.

In some embodiments, a length of the selected genomic nucleotide sequence is more than 3 kb, 3.5 kb, 4 kb, 4.5 kb, 5 kb, 5.5 kb, 6 kb, 6.5 kb, or 7 kb. In some embodiments, the length is about 6574 bp. In some embodiments, the region to be altered is exon 2, exon 3, exon 4, exon 5, and/or exon 6 of mouse IL2RA gene.

In some embodiments, the sequence of the 5′ arm is shown in SEQ ID NO: 5. In some embodiments, the sequence of the 3′ arm is shown in SEQ ID NO: 6.

In some embodiments, the targeting vector further includes a selectable gene marker.

In some embodiments, the target region is derived from human. In some embodiments, the target region is a part or entirety of the nucleotide sequence of a humanized IL2RA. In some embodiments, the nucleotide sequence is shown as one or more of exon 2, exon 3, exon 4, exon 5, and exon 6 of the human IL2RA.

In some embodiments, the nucleotide sequence of the human IL2RA encodes the human IL2RA protein with the NCBI accession number NP_000408.1 (SEQ ID NO: 4). In some emboldens, the nucleotide sequence of the human IL2RA is selected from the nucleotides from the position 6019444 to the position 6026017 of NC_000010.11 (SEQ ID NO: 7).

The disclosure also relates to a cell including the targeting vector as described herein.

The disclosure also relates to a method for establishing a genetically-modified non-human animal expressing two human or chimeric (e.g., humanized) genes. The method includes the steps of

(a) using the method for establishing a IL2RA gene humanized animal model to obtain a IL2RA gene genetically modified humanized mouse;

(b) mating the IL2RA gene genetically modified humanized mouse obtained in step (a) with another humanized mouse, and then screening to obtain a double humanized mouse model.

In some embodiments, in step (b), the IL2RA gene genetically modified humanized mouse obtained in step (a) is mated with a PD-1 humanized mouse to obtain a IL2RA and PD-1 double humanized mouse model.

The disclosure also relates to non-human mammal generated through the methods as described herein.

In some embodiments, the genome thereof contains human gene(s).

In some embodiments, the non-human mammal is a rodent. In some embodiments, the non-human mammal is a mouse.

In some embodiments, the non-human mammal expresses a protein encoded by a humanized IL2RA gene.

The disclosure also relates to an offspring of the non-human mammal.

In another aspect, the disclosure relates to a tumor bearing non-human mammal model, characterized in that the non-human mammal model is obtained through the methods as described herein.

In some embodiments, the non-human mammal is a rodent. In some embodiments, the non-human mammal is a mouse.

The disclosure also relates to a cell (e.g., stem cell or embryonic stem cell) or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

In one aspect, the disclosure relates to a IL2RA amino acid sequence of a humanized mouse, wherein the amino acid sequence is selected from the group consisting of:

a) an amino acid sequence shown in SEQ ID NO: 11;

b) an amino acid sequence having a homology of at least 90% with the amino acid sequence shown in SEQ ID NO: 11;

c) an amino acid sequence encoded by a nucleic acid sequence, wherein the nucleic acid sequence is able to hybridize to a nucleotide sequence encoding the amino acid shown in SEQ ID NO: 11 under a low stringency condition or a strict stringency condition;

d) an amino acid sequence having a homology of at least 90%, 91%, 92%, 93%, 94%, 95%, 96%, 97%, 98%, or at least 99% with the amino acid sequence shown in SEQ ID NO: 11;

e) an amino acid sequence that is different from the amino acid sequence shown in SEQ ID NO: 11 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 11.

The disclosure also relates to a IL2RA nucleic acid sequence of a humanized mouse, wherein the nucleic acid sequence is selected from the group consisting of:

a) a nucleic acid sequence that encodes the IL2RA amino acid sequence of a humanized mouse;

b) a nucleic acid sequence that is set forth in SEQ ID NOS: 8-10 or 12-13;

c) a nucleic acid sequence that can hybridize to the nucleotide sequence as shown in SEQ ID NOS: 8-10 or 12-13 under a low stringency condition or a strict stringency condition;

d) a nucleic acid sequence that has a homology of at least 90%, 91%, 92%, 93%, 94%, 95%, 96%, 97%, 98%, or at least 99% with the nucleotide sequence as shown in SEQ ID NOS: 8-10 or 12-13;

f) a nucleic acid sequence that encodes an amino acid sequence, wherein the amino acid sequence has a homology of at least 90% with the amino acid sequence shown in SEQ ID NO: 11;

g) a nucleic acid sequence that encodes an amino acid sequence, wherein the amino acid sequence has a homology of at least 90%, 91%, 92%, 93%, 94%, 95%, 96%, 97%, 98%, or at least 99% with the amino acid sequence shown in SEQ ID NO: 11;

h) a nucleic acid sequence that encodes an amino acid sequence, wherein the amino acid sequence is different from the amino acid sequence shown in SEQ ID NO: 11 by no more than 10, 9, 8, 7, 6, 5, 4, 3, 2 or no more than 1 amino acid; and/or

i) a nucleic acid sequence that encodes an amino acid sequence, wherein the amino acid sequence comprises a substitution, a deletion and/or insertion of 1, 2, 3, 4, 5, 6, 7, 8, 9, or more amino acids to the amino acid sequence shown in SEQ ID NO: 11.

The disclosure further relates to a IL2RA genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the IL2RA gene function, human IL2RA antibodies, the drugs or efficacies for human IL2RA targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse IL2RA gene locus.

FIG. 1B is a schematic diagram showing human IL2RA gene locus.

FIG. 2 is a schematic diagram showing humanized IL2RA gene locus.

FIG. 3 is a schematic diagram showing an IL2RA gene targeting strategy.

FIG. 4 shows Southern Blot results. WT is wild-type.

FIG. 5 is a schematic diagram showing the FRT recombination process. Positive heterozygous mice were mated with the Flp transgenic mice.

FIG. 6A shows PCR identification result of samples collected from tails of F1 generation mice. Primers WT-F/WT-R were used for amplification. WT is wild-type C57BL/6 mice. H₂O is a blank control. PC, PC1, and PC2 are positive controls. M is marker. F1-1, F1-2, F1-3, F1-4, F1-5, F1-6, F1-7, and F1-8 are labels for mice.

FIG. 6B shows PCR identification result of samples collected from tails of F1 generation mice. Primers WT-F/Mut-R were used for amplification. WT is wild-type C57BL/6 mice. H₂O is a blank control. PC, PC1, and PC2 are positive controls. M is marker. F1-1, F1-2, F1-3, F1-4, F1-5, F1-6, F1-7, and F1-8 are labels for mice.

FIG. 6C shows PCR identification result of samples collected from tails of F1 generation mice. Primers Frt-F/Frt-R were used for amplification. WT is wild-type C57BL/6 mice. H₂O is a blank control. PC, PC1, and PC2 are positive controls. M is marker. F1-1, F1-2, F1-3, F1-4, F1-5, F1-6, F1-7, and F1-8 are labels for mice.

FIG. 6D shows PCR identification result of samples collected from tails of F1 generation mice. Primers Flp-F2/Flp-R2 were used for amplification. WT is wild-type C57BL/6 mice. H₂O is a blank control. PC, PC1, and PC2 are positive controls. M is marker. F1-1, F1-2, F1-3, F1-4, F1-5, F1-6, F1-7, and F1-8 are labels for mice.

FIG. 7A is a graph showing the flow cytometry analysis result of wild-type C57BL/6 mice, wherein cells were stained by mCD25 PE and mCD4 FITC.

FIG. 7B is a graph showing the flow cytometry analysis result of heterozygous (h/+) humanized IL2RA (B-hIL2RA) mice, wherein cells were stained by mCD25 PE and mCD4 FITC.

FIG. 7C is a graph showing the flow cytometry analysis result of wild-type C57BL/6 mice, wherein cells were stained by hCD25 APC and mCD4 FITC.

FIG. 7D is a graph showing the flow cytometry analysis result of heterozygous (h/+) B-hIL2RA mice, wherein cells were stained by hCD25 APC and mCD4 FITC.

FIG. 7E is a graph showing the flow cytometry analysis result of anti-mCD3 antibody-stimulated wild-type C57BL/6 mice, wherein cells were stained by mCD25 PE and mCD4 FITC.

FIG. 7F is a graph showing the flow cytometry analysis result of anti-mCD3 antibody-stimulated heterozygous (h/+) B-hIL2RA mice, wherein cells were stained by mCD25 PE and mCD4 FITC.

FIG. 7G is a graph showing the flow cytometry analysis result of anti-mCD3 antibody-stimulated wild-type C57BL/6 mice, wherein cells were stained by hCD25 APC and mCD4 FITC.

FIG. 7H is a graph showing the flow cytometry analysis result of anti-mCD3 antibody-stimulated heterozygous (h/+) B-hIL2RA mice, wherein cells were stained by hCD25 APC and mCD4 FITC.

FIG. 8 is a set of flow cytometry results showing IL2RA protein expression in NK cells from anti-mCD3 antibody stimulated mouse spleen. B-hIL2RA (h/h) represents IL2RA gene humanized homozygous mice. WT represents wild-type C57BL/6 mice. Cells were stained by PE/Cy™ 7 Mouse anti-mouse NK1.1, PerCP/Cy5.5 anti-mouse TCR β chain, Brilliant Violet 510™ anti-mouse CD45, and either mIL2RA-PE or hIL2RA-APC.

FIG. 9 is a set of flow cytometry results showing IL2RA protein expression in CD4+ T cells from anti-mCD3 antibody stimulated mouse spleen. B-hIL2RA (h/h) represents IL2RA gene humanized homozygous mice. WT represents wild-type C57BL/6 mice. Cells were stained by Brilliant Violet 421™ anti-mouse CD4, PerCP/Cy5.5 anti-mouse TCR β chain, Brilliant Violet 510™ anti-mouse CD45, and either mIL2RA-PE or hIL2RA-APC.

FIG. 10 is a set of flow cytometry results showing IL2RA protein expression in CD8+ T cells from mCD3-stimulated mouse spleen. B-hIL2RA (h/h) represents IL2RA gene humanized homozygous mice. WT represents wild-type C57BL/6 mice. Cells were stained by Brilliant Violet 711™ anti-mouse CD8a, PerCP/Cy5.5 anti-mouse TCR chain, Brilliant Violet 510™ anti-mouse CD45, and either mIL2RA-PE or hIL2RA-APC.

FIG. 11A shows percentage of p-STAT5 in CD3+ lymphocytes that were stimulated by recombinant mouse IL2 at 0 U/mL, 10 U/mL, 100 U/mL, or 1000 U/mL. WT represents wild-type C57BL/6 mouse splenocytes. B-hIL2RA (h/h) represents IL2RA gene humanized homozygous mouse splenocytes. The level of p-STAT5 was measured by flow cytometry.

FIG. 11B shows percentage of p-STAT5 in CD3+ lymphocytes that were stimulated by human IL2 at 0 U/mL, 10 U/mL, 100 U/mL, or 1000 U/mL. WT represents wild-type C57BL/6 mouse splenocytes. B-hIL2RA (h/h) represents IL2RA gene humanized homozygous mouse splenocytes. The level of p-STAT5 was measured by flow cytometry.

FIG. 12 shows percentage of p-STAT5 in CD3+ lymphocytes that were stimulated by recombinant mouse IL2 at 100 U/mL. WT represents wild-type C57BL/6 mouse splenocytes. B-hIL2RA (h/h) represents IL2RA gene humanized homozygous mouse splenocytes. The level of p-STAT5 was measured by flow cytometry. Tab02 and Tab04 were anti-human IL2RA antibodies.

FIG. 13 shows the average weight of humanized IL2RA homozygous mice that were xenografted with mouse colon cancer cells (MC38), and then treated with an control antibody (G1), and anti-IL2RA antibodies AB1 (G2), or AB2 (G3) at 10 mg/kg.

FIG. 14 shows the percentage change of average weight of humanized IL2RA homozygous mice that were xenografted with mouse colon cancer cells (MC38), and then treated with an control antibody (G1), and anti-IL2RA antibodies AB1 (G2), or AB2 (G3) at 10 mg/kg.

FIG. 15 shows the average tumor volume of humanized IL2RA homozygous mice that were xenografted with mouse colon cancer cells (MC38), and then treated with an control antibody (G1), and anti-IL2RA antibodies AB1 (G2), or AB2 (G3) at 10 mg/kg.

FIG. 16 shows the average weight of humanized IL2RA homozygous mice that were xenografted with mouse colon cancer cells (MC38), and then treated with anti-IL2RA antibodies ab1 (G2), or ab2 (G3) at 10 mg/kg. G1 is a control group.

FIG. 17 shows the percentage change of average weight of humanized IL2RA homozygous mice that were xenografted with mouse colon cancer cells (MC38), and then treated with anti-IL2RA antibodies ab1 (G2), or ab2 (G3) at 10 mg/kg. G1 is a control group.

FIG. 18 shows the average tumor volume of humanized IL2RA homozygous mice that were xenografted with mouse colon cancer cells (MC38), and then treated with anti-IL2RA antibodies ab1 (G2), or ab2 (G3) at 10 mg/kg. G1 is a control group.

FIG. 19 shows the alignment between mouse IL2RA amino acid sequence (NP_032393.3; SEQ ID NO: 2) and human IL2RA amino acid sequence (NP_000408.1; SEQ ID NO: 4).

### DETAILED DESCRIPTION

This disclosure relates to transgenic non-human animal with human or chimeric (e.g., humanized) IL2RA, and methods of use thereof.

Interleukin-2 (IL-2) is a cytokine capable of sustaining the proliferative potential of T-lymphocytes. IL-2 was determined to be the primary growth factor in activated T-lymphocytes, which is capable of driving clonal expansion and effector cell maturation. IL-2 stimulation can also lead to the growth of natural killer (NK) cells. IL-2 mediates its biologic effects via the IL-2 receptor (IL-2R) complex. IL-2R is comprised of three distinct subunits: alpha, beta, and gamma chains. IL-2R is related to proliferation of activated CD4-, CD8-, CD4+8+, CD4+, and CD8+ T cells. The importance of the IL2RA is demonstrated by its high affinity for IL-2. Signaling pathways involving IL-2R include Jak3-dependent activation of Jak1, which has been shown to be the key proliferative signal in fibroblasts. The elevated expression of sIL2RA and IL2RA protein in tumor cells has been detected in a vast array of cancers, e.g., lung cancer, melanoma, prostate cancer, esophageal squamous cell carcinoma, leukemia, lymphoma, ovarian cancer, colorectal cancer, and breast cancer. Additionally, elevated mRNA and protein expression of IL2RA has been identified in many solid tumor types, such as ovarian, lung, head-and-neck, and breast. Thus, IL2RA antibodies can be potentially used as cancer therapies.

Experimental animal models are an indispensable research tool for studying the effects of these antibodies (e.g., IL2RA antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullisetal U.S. Pat. No. 4,683,195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wuetal. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N. Y., 1986); each of which is incorporated herein by reference in its entirety.

### IL2RA

IL2RA (interleukin-2 receptor alpha chain, or CD25) is a type I transmembrane protein present on activated T cells, activated B cells, some thymocytes, myeloid precursors, oligodendrocytes, etc. Though IL2RA has been used as a marker to identify CD4+FoxP3+ regulatory T cells in mice, it has been found that a large proportion of resting memory T cells constitutively express IL2RA in humans. IL2RA is expressed in most B-cell neoplasms, some acute nonlymphocytic leukemias, neuroblastomas, mastocytosis and tumor infiltrating lymphocytes. It functions as the receptor for HTLV-1 and is consequently expressed on neoplastic cells in adult T cell lymphoma/leukemia. Its soluble form, called sIL-2R may be elevated in these diseases and is occasionally used to track disease progression.

The interleukin-2 (IL-2) receptor is formed by the α (IL-2RA, CD25), β (IL-2RB, CD122) and γ common (IL-2RG, CD132) subunits, and plays a vital role in maintaining the immune system. The interleukin 2 (IL2) receptor alpha (IL2RA) and beta (IL2RB) chains, together with the common gamma chain (IL2RG), constitute the high-affinity IL2 receptor. Homodimeric alpha chains (IL2RA) result in low-affinity receptor, while homodimeric beta (IL2RB) chains produce a medium-affinity receptor. The high-affinity receptor for IL-2 incorporates all three chains (α, β, and γ) and is present on activated T cells, activated B cells, and Treg cells. The medium-affinity receptor consists of the gamma and beta chains only, and is expressed on NK cells, as well as resting T and B cells. The low-affinity receptor consists of the alpha chain and is expressed on dendritic cells.

Among the IL-2 receptors, IL2RA is a unique subunit that exclusively binds IL-2, while CD132 binds the common γc family cytokines (IL-4, IL-7, IL-9, IL-15 and IL-21), and the CD122 subunit binds IL-15. IL2RA is constitutively expressed at high levels by regulatory T cells (Tregs), and enables them to be the first responders to IL-2 during an immune response and promotes the transcription of FOXP3 by amplifying IL-2 signaling in a STAT5-dependent fashion. Interestingly, single nucleotide polymorphism (SNP) studies of the IL2RA gene have been associated with several forms of autoimmunity demonstrating that IL-2 signaling via IL2RA is an important axis in regulating tolerance. IL2RA is also critical for effector T cell expansion in response to IL-2 immediately after antigenic stimulation.

The formation of the high-affinity quaternary IL-2-IL-2R complex leads to signal transduction through the tyrosine kinases Jak1 and Jak3, which are associated with IL-2Rβ and γc, respectively. Three tyrosine residues within the cytoplasmic tail of IL-2R13 are phosphorylated to promote recruitment of the adaptor Shc (Y338 human; Y341 mouse), leading to activation of the MAPK and PI-3K kinase pathways, and predominately the Stat5 transcription factor (Y392 and Y510 human; Y398 and Y505 mouse), resulting in Stat5-dependent gene regulation. The quaternary IL-2-IL-2R complex is rapidly internalized, where IL-2, IL-2R13, and γc are rapidly degraded, but IL-2Ra is recycled to the cell surface. Thus, those functional activities that require sustained IL-2R signaling require a continued source of IL-2 to engage IL-2Ra and form additional IL-2-IL-2R signaling complexes.

IL2RA is a protein that in humans is encoded by the IL2RA gene. Normally an integral-membrane protein, soluble IL2RA has been isolated and determined to result from extracellular proteolysis. Alternately-spliced IL2RA mRNAs have been isolated, but the significance of each is currently unknown. Mutations in this gene are associated with interleukin 2 receptor alpha deficiency.

A detailed description of IL2RA and its function can be found, e.g., in Goudy, Kevin, et al., “Human IL2RA null mutation mediates immunodeficiency with lymphoproliferation and autoimmunity.” Clinical Immunology 146.3 (2013): 248-261; Kuhn, Deborah J., and Q. Ping Dou. “The role of interleukin-2 receptor alpha in cancer.” Front Biosci 10 (2005): 1462-1474; Malek, Thomas R., and Iris Castro. “Interleukin-2 receptor signaling: at the interface between tolerance and immunity.” Immunity 33.2 (2010): 153-165; and Jiang et al., “Role of IL-2 in cancer immunotherapy.” Oncoimmunology 5.6 (2016): e1163462; each of which is incorporated by reference in its entirety.

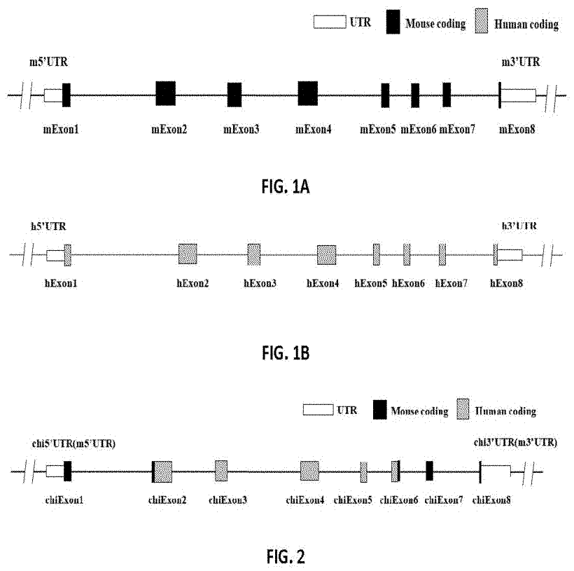

In human genomes, IL2RA gene (Gene ID: 3559) locus has eight exons, exon 1, exon 2, exon 3, exon 4, exon 5, exon 6, exon 7, and exon 8 (FIG. 1B). The IL2RA protein also has an extracellular region, a transmembrane region, and a cytoplasmic region, and the signal peptide is located at the extracellular region of IL2RA. The nucleotide sequence for human IL2RA mRNA is NM_000417.2 (SEQ ID NO: 3), and the amino acid sequence for human IL2RA is NP_000408.1 (SEQ ID NO: 4). The location for each exon and each region in human IL2RA nucleotide sequence and amino acid sequence is listed below:

TABLE 1

| Human IL2RA | NM_000417.2 | NP_000408.1 |
|---|---|---|
| (approximate | 3216bp | 272aa |
| location) | SEQ ID NO: 3 | SEQIDNO: 4 |
| Exon 1 | 1-283 | 1-21 |
| Exon 2 | 284-475 | 22-85 |
| Exon 3 | 476-586 | 86-122 |
| Exon 4 | 587-802 | 123-194 |
| Exon 5 | 803-874 | 195-218 |
| Exon 6 | 875-946 | 219-242 |
| Exon 7 | 947-1013 | 243-264 |
| Exon 8 | 1014-3216 | 265-272 |
| Signal peptide | 220-282 | 1-21 |
| Extracellular | 283-939 | 22-240 |
| Transmembrane region | 940-996 | 241-259 |
| Cytoplasmic | 997-1035 | 260-272 |
| Donor region in Example | 292-930 | 25-237 |

In mice, IL2RA gene locus has eight exons, exon 1, exon 2, exon 3, exon 4, exon 5, exon 6, exon 7, and exon 8 (FIG. 1A). The mouse IL2RA protein also has an extracellular region, a transmembrane region, and a cytoplasmic region, and the signal peptide is located at the extracellular region of IL2RA. The nucleotide sequence for mouse IL2RA mDNA is NM_008367.3 (SEQ ID NO: 1), the amino acid sequence for mouse IL2RA is NP_032393.3 (SEQ ID NO: 2). The location for each exon and each region in the mouse IL2RA nucleotide sequence and amino acid sequence is listed below:

TABLE 2

| | NM_008367.3 | NP_032393.3 |
|---|---|---|
| Mouse IL2RA | 4428bp | 268aa |
| (approximate location) | SEQ ID NO: 1 | SEQIDNO: 2 |
| Exon 1 | 1-273 | 1-21 |
| Exon 2 | 274-450 | 22-80 |
| Exon 3 | 451-564 | 81-118 |
| Exon 4 | 565-780 | 119-190 |
| Exon 5 | 781-852 | 191-214 |
| Exon 6 | 853-924 | 215-238 |
| Exon 7 | 925-991 | 239-260 |
| Exon 8 | 992-4428 | 261-268 |
| Signal peptide | 210-272 | 1-21 |
| Extracellular | 273-917 | 22-236 |
| Transmembrane region | 918-980 | 237-257 |
| Cytoplasmic | 981-1013 | 258-268 |
| Replaced region in Example | 282-908 | 25-233 |

The mouse IL2RA gene (Gene ID: 16168) is located in Chromosome 2 of the mouse genome, which is located from 11642792 to 11693194 of NC_000068.7 (GRCm38.p4 (GCF_000001635.24)). The 5′-UTR is from 11,693,193 to 11,689,784, exon 1 is from 11,689,783 to 11,689,758, the first intron is from 11,689,757 to 11,684,444, exon 2 is from 11,684,443 to 11,684,377, the second intron is from 11,684,376 to 11,683,126, exon 3 is from 11,683,125 to 11,683,054, the third intron is from 11,683,053 to 11,682,003, exon 4 is from 11,682,002 to 11,681,931, the fourth intron is from 11,681,930 to 11,580,426, exon 5 is from 11,580,425 to 11,680,210, the fifth intron is from 11,680,209 to 11,676,941, exon 6 is from 11,676,940 to 11,676,827, the sixth intron is from 11,676,826 to 11,674,472, exon 7 is from 11,674,471 to 11,674,295, the seventh intron is from 11,674,294 to 11,643,065, exon 8 is from 11,643,064 to 11,642,807, and the 3′-UTR is from 11,643,001 to 11,642,807, based on transcript NM_008367.3. All relevant information for mouse IL2RA locus can be found in the NCBI website with Gene ID: 16184, which is incorporated by reference herein in its entirety.

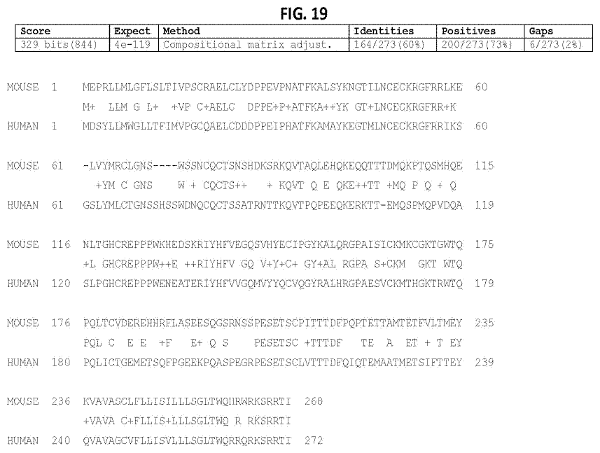

FIG. 19 shows the alignment between mouse IL2RA amino acid sequence (NP_032393.3; SEQ ID NO: 2) and human IL2RA amino acid sequence (NP_000408.1; SEQ ID NO: 4). Thus, the corresponding amino acid residue or region between human and mouse IL2RA can be found in FIG. 19.

IL2RA genes, proteins, and locus of the other species are also known in the art. For example, the gene ID for IL2RA in Rattus norvegicus (rat) is 25704, the gene ID for IL2RA in Macaca mulatta (Rhesus monkey) is 574300, the gene ID for IL2RA in Canis lupus familiaris (dog) is 403870, and the gene ID for IL2RA in Sus scrofa (pig) is 396814. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) IL2RA nucleotide sequence and/or amino acid sequences. In some embodiments, the entire sequence of mouse exon 1, exon 2, exon 3, exon 4, exon 5, exon 6, exon 7, exon 8, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, exon 8, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, signal peptide, extracellular region, transmembrane region, or cytoplasmic region. In some embodiments, a region, a portion, or the entire sequence of mouse exon 1, exon 2, exon 3, exon 4, exon 5, exon 6, exon 7, and/or exon 8 (e.g., exon 2, exon 3, exon 4, exon 5, and exon 6) are replaced by a region, a portion, or the entire sequence of the human exon 1, exon 2, exon 3, exon 4, exon 5, exon 6, exon 7, and/or exon 8 (e.g., exon 2, exon 3, exon 4, exon 5, and exon 6) sequence.

In some embodiments, the present disclosure also provides a chimeric (e.g., humanized) IL2RA nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL2RA mRNA sequence (e.g., SEQ ID NO: 1), mouse IL2RA amino acid sequence (e.g., SEQ ID NO: 2), or a portion thereof (e.g., a portion of exon 2, exon 3, exon 4, exon 5, and a portion of exon 6); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL2RA mRNA sequence (e.g., SEQ ID NO: 3), human IL2RA amino acid sequence (e.g., SEQ ID NO: 4), or a portion thereof (e.g., a portion of exon 2, exon 3, exon 4, exon 5, and a portion of exon 6).

In some embodiments, the sequence encoding amino acids 25-233 of mouse IL2RA (SEQ ID NO: 2) is replaced. In some embodiments, the sequence is replaced by a sequence encoding a corresponding region of human IL2RA (e.g., amino acids 25-237 of human IL2RA (SEQ ID NO: 4)).

In some embodiments, the sequence encoding amino acids 22-236 of mouse IL2RA (SEQ ID NO: 2) is replaced. In some embodiments, the sequence is replaced by a sequence encoding a corresponding region of human IL2RA (e.g., amino acids 22-240 of human IL2RA (SEQ ID NO: 4)).

In some embodiments, the nucleic acids as described herein are operably linked to a promotor or regulatory element, e.g., an endogenous mouse IL2RA promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse IL2RA nucleotide sequence (e.g., exon 2, exon 3, exon 4, exon 5, exon 6, or NM_008367.3 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse IL2RA nucleotide sequence (e.g., exon 2, exon 3, exon 4, exon 5, exon 6, or NM_008367.3 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human IL2RA nucleotide sequence (e.g., exon 2, exon 3, exon 4, exon 5, exon 6, or NM_000417.2 (SEQ ID NO: 3)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human IL2RA nucleotide sequence (e.g., exon 2, exon 3, exon 4, exon 5, exon 6, or NM_000417.2 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse IL2RA amino acid sequence (e.g., amino acids encoded by exon 2, exon 3, exon 4, exon 5, and/or exon 6 of NM_008367.3 (SEQ ID NO: 1); or NP_032393.3 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse IL2RA amino acid sequence (e.g., amino acids encoded by exon 2, exon 3, exon 4, exon 5, and/or exon 6 of NM_008367.3 (SEQ ID NO: 1); or NP_032393.3 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human IL2RA amino acid sequence (e.g., amino acids encoded by exon 2, exon 3, exon 4, exon 5, and/or exon 6 of NM_000417.2 (SEQ ID NO: 3); or NP_000408.1 (SEQ ID NO: 4)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human IL2RA amino acid sequence (e.g., amino acids encoded by exon 2, exon 3, exon 4, exon 5, and/or exon 6 of NM_000417.2 (SEQ ID NO: 3); or NP_000408.1 (SEQ ID NO: 4)).

The present disclosure also provides a humanized IL2RA mouse amino acid sequence, wherein the amino acid sequence is selected from the group consisting of:

a) an amino acid sequence shown in SEQ ID NO: 11;

b) an amino acid sequence having a homology of at least 90% with or at least 90% identical to the amino acid sequence shown in SEQ ID NO: 11;

c) an amino acid sequence encoded by a nucleic acid sequence, wherein the nucleic acid sequence is able to hybridize to a nucleotide sequence encoding the amino acid shown in SEQ ID NO: 11 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 11;

e) an amino acid sequence that is different from the amino acid sequence shown in SEQ ID NO: 11 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 11.

The present disclosure also relates to a IL2RA nucleic acid (e.g., DNA or RNA) sequence, wherein the nucleic acid sequence can be selected from the group consisting of:

a) a nucleic acid sequence as shown in SEQ ID NO: 10, or a nucleic acid sequence encoding a homologous IL2RA amino acid sequence of a humanized mouse IL2RA;

b) a nucleic acid sequence that is able to hybridize to the nucleotide sequence as shown in SEQ ID NO: 10 under a low stringency condition or a strict stringency condition;

c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 10;

d) a nucleic acid sequence that encodes an amino acid sequence, wherein the amino acid sequence has a homology of at least 90% with or at least 90% identical to the amino acid sequence shown in SEQ ID NO: 11;

e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 11;

f) a nucleic acid sequence that encodes an amino acid sequence, wherein the amino acid sequence is different from the amino acid sequence shown in SEQ ID NO: 11 by no more than 10, 9, 8, 7, 6, 5, 4, 3, 2 or no more than 1 amino acid; and/or

g) a nucleic acid sequence that encodes an amino acid sequence, wherein the amino acid sequence comprises a substitution, a deletion and/or insertion of one or more amino acids to the amino acid sequence shown in SEQ ID NO: 11.

The present disclosure further relates to a IL2RA genomic DNA sequence of a humanized mouse. The DNA sequence is obtained by a reverse transcription of the mRNA obtained by transcription thereof is consistent with or complementary to the DNA sequence homologous to the sequence shown in SEQ ID NO:10.

The disclosure also provides an amino acid sequence that has a homology of at least 90% with, or at least 90% identical to the sequence shown in SEQ ID NO: 11, and has protein activity. In some embodiments, the homology with the sequence shown in SEQ ID NO: 11 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 11 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 10, and encodes a polypeptide that has protein activity. In some embodiments, the homology with the sequence shown in SEQ ID NO: 10 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 10 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) IL2RA from an endogenous non-human IL2RA locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous IL2RA locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized IL2RA gene or a humanized IL2RA nucleic acid. In some embodiments, at least one or more portions of the gene or the nucleic acid is from the human IL2RA gene, at least one or more portions of the gene or the nucleic acid is from a non-human IL2RA gene. In some embodiments, the gene or the nucleic acid comprises a sequence that encodes an IL2RA protein. The encoded IL2RA protein is functional or has at least one activity of the human IL2RA protein or the non-human IL2RA protein, e.g., binding or interacting with human or non-human IL2, IL2RB (CD122), and/or IL2RG (CD132); specific binding with human or non-human IL2 to trigger downstream signaling cascade; stimulating IL2-mediated cell proliferation; promoting T cell growth; promoting B cell proliferation and differentiation; enhancing the killing activity of cytotoxic T lymphocytes, natural killer cells, and/or lymphokine-activated killer cells; inducing the production of other cytokines and expression of cytokine receptors; regulating immune tolerance by controlling regulatory T cells (Tregs); amplifying IL2 signaling; inducing effector T cell expansion; regulating MAPK and PI-3K kinase pathways, RET signaling pathway, PEDF-induced signaling pathways and/or TGF-β signaling pathways; regulating apoptotic pathways in synovial fibroblasts; regulating Th17 cell differentiation; and/or upregulating the immune response.

In some embodiments, the chimeric protein or the chimeric polypeptide is a humanized IL2RA protein or a humanized IL2RA polypeptide. In some embodiments, at least one or more portions of the amino acid sequence of the protein or the polypeptide is from a human IL2RA protein, and at least one or more portions of the amino acid sequence of the protein or the polypeptide is from a non-human IL2RA protein. The humanized IL2RA protein or the humanized IL2RA polypeptide is functional or has at least one activity of the human IL2RA protein or the non-human IL2RA protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 12951/SV, 12951/SvIm), 129S2, 129S4, 129S5, 12959/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized IL2RA animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γcnull mice (Ito, M. et al., NOD/SCID/γcnull mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human IL2RA locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature IL2RA coding sequence with human mature IL2RA coding sequence. In some embodiments, the mature IL2RA is the soluble IL2RA as described herein.

Genetically modified non-human animals that comprise a modification of an endogenous non-human IL2RA locus. In some embodiments, the modification can comprise a human nucleic acid sequence encoding at least a portion of a mature IL2RA protein (e.g., at least 10%, 20%, 30%, 40%, 50%, 60%, 70%, 80%, 90%, 95%, 96%, 97%, 98%, or 99% identical to the mature IL2RA protein sequence). Although genetically modified cells are also provided that can comprise the modifications described herein (e.g., ES cells, somatic cells), in many embodiments, the genetically modified non-human animals comprise the modification of the endogenous IL2RA locus in the germline of the animal.

Genetically modified animals can express a human IL2RA and/or a chimeric (e.g., humanized) IL2RA from endogenous mouse loci, wherein the endogenous mouse IL2RA gene has been replaced with a human IL2RA gene and/or a nucleotide sequence that encodes a region of human IL2RA sequence or an amino acid sequence that is at least 10%, 20%, 30%, 40%, 50%, 60%, 70&, 80%, 90%, 95%, 96%, 97%, 98%, or 99% identical to the human IL2RA sequence. In various embodiments, an endogenous non-human IL2RA locus is modified in whole or in part to comprise human nucleic acid sequence encoding at least one protein-coding sequence of a mature IL2RA protein.

In some embodiments, the genetically modified mice express the human IL2RA and/or chimeric IL2RA (e.g., humanized IL2RA) from endogenous loci that are under control of mouse promoters and/or mouse regulatory elements. The replacement(s) at the endogenous mouse loci provide non-human animals that express human IL2RA or chimeric IL2RA (e.g., humanized IL2RA) in appropriate cell types and in a manner that does not result in the potential pathologies observed in some other transgenic mice known in the art. The human IL2RA or the chimeric IL2RA (e.g., humanized IL2RA) expressed in animal can maintain one or more functions of the wild-type mouse or human IL2RA in the animal. For example, human or non-human IL2RA ligands (e.g., IL2) can bind to the expressed IL2RA, upregulate immune response, e.g., upregulate immune response by at least 10%, 20%, 30%, 40%, or 50%. Furthermore, in some embodiments, the animal does not express endogenous IL2RA. In some embodiments, the animal expresses a decreased level of endogenous IL2RA as compared to a wild-type animal. As used herein, the term “endogenous IL2RA” refers to IL2RA protein that is expressed from an endogenous IL2RA nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human IL2RA (NP_000408.1) (SEQ ID NO: 4). In some embodiments, the genome comprises a sequence encoding an amino acid sequence that is at least 70%, 75%, 80%, 85%, 90%, 95%, 99%, or 100% identical to SEQ ID NO: 11.

The genome of the genetically modified animal can comprise a replacement at an endogenous IL2RA gene locus of a sequence encoding a region of endogenous IL2RA with a sequence encoding a corresponding region of human IL2RA. In some embodiments, the sequence that is replaced is any sequence within the endogenous IL2RA gene locus, e.g., exon 1, exon 2, exon 3, exon 4, exon 5, exon 6, exon 7, exon 8, 5′-UTR, 3′-UTR, the first intron, the second intron, and the third intron, the fourth intron, the fifth intron, the sixth intron, the seventh intron, etc. In some embodiments, the sequence that is replaced is within the regulatory region of the endogenous IL2RA gene. In some embodiments, the sequence that is replaced is exon 2, exon 3, exon 4, exon 5, exon 6, or a portion thereof, of an endogenous mouse IL2RA gene locus.

The genetically modified animal can have one or more cells expressing a human or chimeric IL2RA (e.g., humanized IL2RA) having an extracellular region and a cytoplasmic region, wherein the extracellular region comprises a sequence that is at least 50%, 60%, 70%, 80%, 90%, 95%, 99% identical to the extracellular region of human IL2RA. In some embodiments, the extracellular region of the humanized IL2RA has a sequence that has at least 10, 20, 30, 40, 50, 60, 70, 80, 90, 100, 110, 120, 130, 140, 150, 160, 170, or 180 amino acids (e.g., contiguously or non-contiguously) that are identical to human IL2RA. Because human IL2RA and non-human IL2RA (e.g., mouse IL2RA) sequences, in many cases, are different, antibodies that bind to human IL2RA will not necessarily have the same binding affinity with non-human IL2RA or have the same effects to non-human IL2RA. Therefore, the genetically modified animal having a human or a humanized extracellular region can be used to better evaluate the effects of anti-human IL2RA antibodies in an animal model. In some embodiments, the genome of the genetically modified animal comprises a sequence encoding an amino acid sequence that corresponds to a portion or the entire sequence of exon 2, exon 3, exon 4, exon 5, and/or exon 6 of human IL2RA, a portion or the entire sequence of extracellular region of human IL2RA (with or without signal peptide), or a portion or the entire sequence of amino acids 25-237 of SEQ ID NO: 4.

In some embodiments, the non-human animal can have, at an endogenous IL2RA gene locus, a nucleotide sequence encoding a chimeric human/non-human IL2RA polypeptide, wherein a human portion of the chimeric human/non-human IL2RA polypeptide comprises a portion of human IL2RA extracellular domain, and wherein the animal expresses a functional IL2RA on a surface of a cell of the animal. The human portion of the chimeric human/non-human IL2RA polypeptide can comprise a portion of exon 2, exon 3, exon 4, exon 5, and/or exon 6 of human IL2RA. In some embodiments, the human portion of the chimeric human/non-human IL2RA polypeptide can comprise a sequence that is at least 80%, 85%, 90%, 95%, or 99% identical to amino acids 25-237 of SEQ ID NO: 4.

In some embodiments, the non-human portion of the chimeric human/non-human IL2RA polypeptide comprises transmembrane and/or cytoplasmic regions of an endogenous non-human IL2RA polypeptide.

Furthermore, the genetically modified animal can be heterozygous with respect to the replacement at the endogenous IL2RA locus, or homozygous with respect to the replacement at the endogenous IL2RA locus.

In some embodiments, the humanized IL2RA locus lacks a human IL2RA 5′-UTR. In some embodiment, the humanized IL2RA locus comprises a rodent (e.g., mouse) 5′-UTR. In some embodiments, the humanization comprises a human 3′-UTR. In some embodiments, the humanization comprises a mouse 3′-UTR. In appropriate cases, it may be reasonable to presume that the mouse and human IL2RA genes appear to be similarly regulated based on the similarity of their 5′-flanking sequence. As shown in the present disclosure, humanized IL2RA mice that comprise a replacement at an endogenous mouse IL2RA locus, which retain mouse regulatory elements but comprise a humanization of IL2RA encoding sequence, do not exhibit pathologies. Both genetically modified mice that are heterozygous or homozygous for humanized IL2RA are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized IL2RA gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized IL2RA in the genome of the animal.

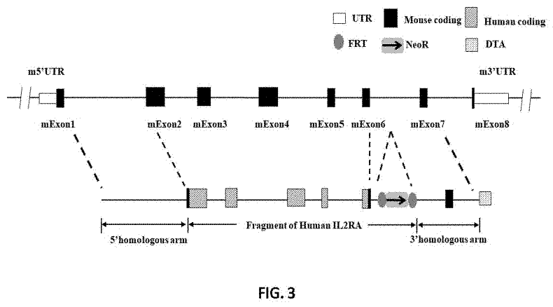

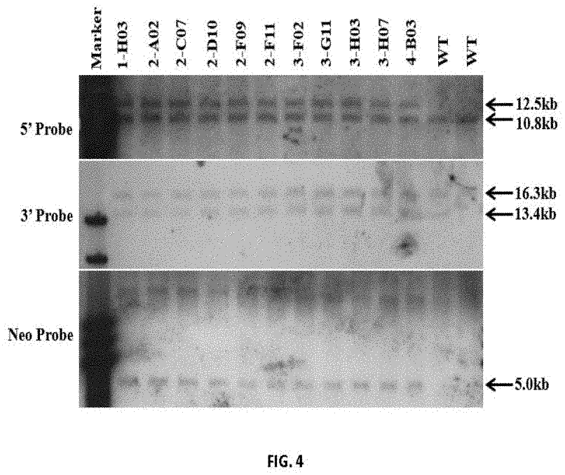

In some embodiments, the non-human mammal comprises the genetic construct as described herein (e.g., gene construct as shown in FIG. 2 or FIG. 3). In some embodiments, a non-human mammal expressing human or humanized IL2RA is provided. In some embodiments, the tissue-specific expression of human or humanized IL2RA protein is provided.

In some embodiments, the expression of human or humanized IL2RA in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human IL2RA protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized IL2RA protein.

### Vectors

The present disclosure relates to a targeting vector, comprising: a) a DNA fragment homologous to the 5′ end of a region to be altered (5′ arm), which is selected from the IL2RA gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL2RA gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000068.7; c) the DNA fragment homologous to the 3′ end of the region to be altered (3′ arm) is selected from the nucleotide sequences that have at least 90% homology to the NCBI accession number NC_000068.7.

In some embodiments, a) the DNA fragment homologous to the 5′ end of a region to be altered (5′ arm) is selected from the nucleotides from the position 11670417 to the position 11674302 of the NCBI accession number NC_000068.7; c) the DNA fragment homologous to the 3′ end of the region to be altered (3′ arm) is selected from the nucleotides from the position 11683554 to the position 11689329 of the NCBI accession number NC_000068.7.

In some embodiments, the length of the selected genomic nucleotide sequence in the targeting vector can be more than about 3 kb, about 3.5 kb, about 4 kb, about 4.5 kb, about 5 kb, about 5.5 kb, about 6 kb, about 6.5 kb, or about 7 kb.

In some embodiments, the region to be altered is exon 1, exon 2, exon 3, exon 4, exon 5, exon 6, exon 7, and/or exon 8 of IL2RA gene (e.g., exon 2, exon 3, exon 4, exon 5, and/or exon 6 of mouse IL2RA gene).

The targeting vector can further include a selected gene marker.

In some embodiments, the sequence of the 5′ arm is shown in SEQ ID NO: 5; and the sequence of the 3′ arm is shown in SEQ ID NO: 6.

In some embodiments, the sequence is derived from human (e.g., 6019444-6026017 of NC_000010.11). For example, the target region in the targeting vector is a part or entirety of the nucleotide sequence of a human IL2RA, preferably exon 2, exon 3, exon 4, exon 5, and/or exon 6 of the human IL2RA. In some embodiments, the nucleotide sequence of the humanized IL2RA encodes the entire or the part of human IL2RA protein with the NCBI accession number NP_000408.1 (SEQ ID NO: 4).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous IL2RA gene locus, a sequence encoding a region of an endogenous IL2RA with a sequence encoding a corresponding region of human or chimeric IL2RA. In some embodiments, the replacement occurs in a germ cell, a somatic cell, a blastocyst, or a fibroblast, etc. The nucleus of a somatic cell or the fibroblast can be inserted into an enucleated oocyte.

FIG. 3 shows a humanization strategy for a mouse IL2RA locus. In FIG. 3, the targeting strategy involves a vector comprising the 5′ end homologous arm, human IL2RA gene fragment, 3′ homologous arm. The process can involve replacing endogenous IL2RA sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous IL2RA sequence with human IL2RA sequence.

Thus, in some embodiments, the methods for making a genetically modified, humanized animal, can include the step of replacing at an endogenous IL2RA locus (or site), a nucleic acid encoding a sequence encoding a region of endogenous IL2RA with a sequence encoding a corresponding region of human IL2RA. The sequence can include a region (e.g., a part or the entire region) of exon 1, exon 2, exon 3, exon 4, exon 5, exon 6, exon 7, and/or exon 8 of a human IL2RA gene. In some embodiments, the sequence includes a region of exon 2, exon 3, exon 4, exon 5, and exon 6 of a human IL2RA gene (e.g., amino acids 25-237 of SEQ ID NO: 4). In some embodiments, the region is located within the extracellular region of IL2RA (e.g., amino acids 22-240 of SEQ ID NO: 4). In some embodiments, the endogenous IL2RA locus is exon 2, exon 3, exon 4, exon 5, and/or exon 6 of mouse IL2RA.

In some embodiments, the methods of modifying a IL2RA locus of a mouse to express a chimeric human/mouse IL2RA peptide can include the steps of replacing at the endogenous mouse IL2RA locus a nucleotide sequence encoding a mouse IL2RA with a nucleotide sequence encoding a human IL2RA, thereby generating a sequence encoding a chimeric human/mouse IL2RA.

In some embodiments, the nucleotide sequence encoding the chimeric human/mouse IL2RA can include a first nucleotide sequence encoding an extracellular region of mouse IL2RA (with or without the mouse or human signal peptide sequence); a second nucleotide sequence encoding an extracellular region of human IL2RA; a third nucleotide sequence encoding a transmembrane and a cytoplasmic region of a mouse IL2RA.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a IL2RA gene humanized animal model, involving the following steps:

(a) providing the cell (e.g. a fertilized egg cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized IL2RA protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized IL2RA, which are useful for testing agents that can decrease or block the interaction between IL2RA and IL2RA ligands (e.g., IL2) or the interaction between IL2RA and anti-human IL2RA antibodies, testing whether an agent can increase or decrease the immune response, and/or determining whether an agent is an IL2RA agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor). In some embodiments, the anti-IL2RA antibody is basiliximab or daclizumab. In some embodiments, the anti-IL2RA antibody blocks or inhibits the IL-2 pathway.

In some embodiments, the genetically modified animals can be used for determining effectiveness of an anti-IL2RA antibody for the treatment of cancer. The methods involve administering the anti-IL2RA antibody (e.g., anti-human IL2RA antibody) to the animal as described herein, wherein the animal has a tumor; and determining the inhibitory effects of the anti-IL2RA antibody to the tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT. Without wishing to be bound by a particular theory, it is believed that in some embodiments, these anti-IL2RA antibodies can target regulatory T cells (Treg) and kill or inhibit the function of Treg cells, as IL2RA is highly expressed on Treg cells, thereby increasing the immune response. In addition, a delicate balance is required for these antibodies, as IL2RA is also expressed on many other cells. Thus, it is important that the humanized IL2RA functions in a largely similar way as compared to the endogenous IL2RA, so that the results in the humanized animals can be used to predict the efficacy or toxicity of these therapeutic agents in the human.

In some embodiments, the tumor comprises one or more cancer cells (e.g., human or mouse cancer cells) that are injected into the animal. In some embodiments, the anti-IL2RA antibody, or anti-IL2 antibody prevents IL2 from binding to IL2RA. In some embodiments, the anti-IL2RA antibody or anti-IL2 antibody does not prevent IL2 from binding to IL2RA.

In some embodiments, the genetically modified animals can be used for determining whether an anti-IL2RA antibody is a IL2RA agonist or antagonist. In some embodiments, the methods as described herein are also designed to determine the effects of the agent (e.g., anti-IL2RA antibodies) on IL2RA, e.g., whether the agent can stimulate immune cells or inhibit immune cells (e.g., T cells, B cells, or NK cells), whether the agent can increase or decrease the production of cytokines, whether the agent can activate or deactivate immune cells (e.g., T cells, B cells, or NK cells), whether the agent can upregulate the immune response or downregulate immune response, and/or whether the agent can induce complement mediated cytotoxicity (CMC) or antibody dependent cellular cytoxicity (ADCC). In some embodiments, the genetically modified animals can be used for determining the effective dosage of a therapeutic agent for treating a disease in the subject, e.g., cancer, or autoimmune diseases.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the anti-IL2RA antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the anti-IL2RA antibody is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the anti-IL2RA antibody is designed for treating hepatocellular, ovarian, colon, or cervical carcinomas. In some embodiments, the anti-IL2RA antibody is designed for treating advanced breast cancer, advanced ovarian cancer, and/or advanced refractory solid tumor. In some embodiments, the anti-IL2RA antibody is designed for treating metastatic solid tumors, NSCLC, melanoma, non-Hodgkin lymphoma, colorectal cancer, and multiple myeloma. In some embodiments, the anti-IL2RA antibody is designed for treating melanoma, pancreatic carcinoma, mesothelioma, hematological malignancies (e.g., Non-Hodgkin's lymphoma, lymphoma, chronic lymphocytic leukemia), or solid tumors (e.g., advanced solid tumors). In some embodiments, the anti-IL2RA antibody is designed for treating carcinomas (e.g., nasopharynx carcinoma, bladder carcinoma, cervix carcinoma, kidney carcinoma or ovary carcinoma).

In some embodiments, the IL2RA antibody is designed for treating acute lymphoblastic leukemia (ALL), B-cell chronic lymphocytic leukemia (B-CLL), hairy cell leukemia (HCL), solid tumors, colorectal cancer, ovarian cancer, prostate cancer, melanoma, lung cancer, breast cancer, gastric cancer, esophageal squamous cell carcinoma (ESCC), leukemia, lymphoma, multiple myeloma, sarcoma, and/or head-and-neck cancer.

In some embodiments, the anti-IL2RA antibody is designed for treating various autoimmune diseases, including multiple sclerosis, rheumatoid arthritis, juvenile idiopathic arthritis, type 1 diabetes, aplastic anemia, asthma, idiopathic thrombocytopenia, uveitis, and eczema. Thus, the methods as described herein can be used to determine the effectiveness of an anti-IL2RA antibody in inhibiting immune response.

In some embodiments, the anti-IL2RA antibody is designed for treating other diseases or disorders, e.g., microbial infection and allergic disorders. In some embodiments, the anti-IL2RA antibody is designed for reducing rejection effects after organ (e.g., kidney) transplantation.

The present disclosure also provides methods of determining toxicity of an antibody (e.g., anti-IL2RA antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the IL2RA gene function, human IL2RA antibodies, drugs for human IL2RA targeting sites, the drugs or efficacies for human IL2RA targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the IL2RA gene humanized non-human animal prepared by the methods described herein, the IL2RA gene humanized non-human animal described herein, the double- or multi-humanized non-human animal generated by the methods described herein (or progeny thereof), a non-human animal expressing the human or humanized IL2RA protein, or the tumor-bearing or inflammatory animal models described herein. In some embodiments, the TCR-T, CAR-T, and/or other immunotherapies can treat the IL2RA-associated diseases described herein. In some embodiments, the TCA-T, CAR-T, and/or other immunotherapies provides an evaluation method for treating the IL2RA-associated diseases described herein.

Genetically Modified Animal Model with Two or More Human or Chimeric Genes

The present disclosure further relates to methods for generating genetically modified animal model with two or more human or chimeric genes. The animal can comprise a human or chimeric IL2RA gene and a sequence encoding an additional human or chimeric protein.

In some embodiments, the additional human or chimeric protein can be programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (TNFRSF4 or OX40).

The methods of generating genetically modified animal model with two or more human or chimeric genes (e.g., humanized genes) can include the following steps:

(a) using the methods of introducing human IL2RA gene or chimeric IL2RA gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric PD-1, CTLA-4, LAG-3, BTLA, PD-L1, CD27, CD28, CD47, CD137, CD154, TIGIT, TIM-3, GITR, SIRPα, or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the IL2RA humanization is directly performed on a genetically modified animal having a human or chimeric PD-1, CTLA-4, BTLA, PD-L1, CD27, CD28, CD47, CD137, CD154, TIGIT, TIM-3, GITR, SIRPα, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-IL2RA antibody and an additional therapeutic agent for the treatment of cancer. The methods include administering the anti-IL2RA antibody and the additional therapeutic agent to the animal, wherein the animal has a tumor; and determining the inhibitory effects of the combined treatment to the tumor. In some embodiments, the additional therapeutic agent is an antibody that specifically binds to IL2, CD122, CD132, PD-1, CTLA-4, BTLA, PD-L1, CD27, CD28, CD47, CD137, CD154, TIGIT, TIM-3, GITR, SIRPα, or OX40. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancer as described herein, e.g., melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, the combination treatment is designed for treating metastatic solid tumors, NSCLC, melanoma, B-cell non-Hodgkin lymphoma, colorectal cancer, and multiple myeloma. In some embodiments, the combination treatment is designed for treating melanoma, carcinomas (e.g., pancreatic carcinoma), mesothelioma, hematological malignancies (e.g., Non-Hodgkin's lymphoma, lymphoma, chronic lymphocytic leukemia), or solid tumors (e.g., advanced solid tumors). In some embodiments, combination treatment is designed for treating acute lymphoblastic leukemia (ALL), B-cell chronic lymphocytic leukemia (B-CLL), hairy cell leukemia (HCL), solid tumors, colorectal cancer, ovarian cancer, prostate cancer, melanoma, lung cancer, breast cancer, gastric cancer, esophageal squamous cell carcinoma (ESCC), leukemia, lymphoma, multiple myeloma, sarcoma, and/or head-and-neck cancer.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

Materials and Methods The following materials were used in the following examples.

StuI, NdeI and DraIII restriction enzymes were purchased from NEB, with the catalog numbers R0187M, R0111S and R3510S respectively.

C57BL/6 mice and Flp transgenic mice were purchased from the China Food and Drugs Research Institute National Rodent Experimental Animal Center.

Flow cytometer was purchased from BD Biosciences (model: FACS Calibur™).

PE anti-mouse CD25 Antibody (mCD25 PE) was purchased from BioLegend, Inc. (catalog number: 102008).

PerCP/Cyanine5.5 anti-mouse TCRβ chain (mTCRβ-PerCP/Cy5.5) was purchased from BioLegend, Inc. (catalog number: 109228).

FITC anti-mouse CD4 Antibody (mCD4 FITC) was purchased from BioLegend, Inc. (catalog number: 116004).

APC anti-human CD25 Antibody (hCD25 APC) was purchased from BioLegend, Inc. (catalog number: 302610).

PE/Cy™ 7 Mouse anti-mouse NK1.1 was purchased from BioLegend, Inc. (catalog number: 552878).

Brilliant Violet 510™ anti-mouse CD45, Brilliant Violet 421™ anti-mouse CD4, and Brilliant Violet 711™ anti-mouse CD8a were purchased from BioLegend, Inc. with catalog numbers 103138, 100438, and 100759, respectively.

Mouse recombinant interleukin 2 (mIL2) was purchased from PeproTech, Inc. (catalog number: 212-12).

Human IL2 (hIL2) was obtained from Jiangsu Hengrui Medicine Co., Ltd.

APC Rat Anti-Mouse CD3 Molecular Complex was purchased from BD Biosciences with catalog number 565643.

Phospho-Stat5 (Tyr694) (D47E7) XP® Rabbit mAb was purchased from Cell Signaling Technology (catalog number: 4322S).

Anti-rabbit IgG (H+L), F(ab′)2 Fragment (Alexa Fluor® 488 Conjugate) was purchased from Cell Signaling Technology (catalog number: 4412).

Human IgG was obtained from Biointron Biological Inc.

Purified NA/LE Hamster anti-mouse CD3e (mCD3) antibody was purchased from BD Biosciences (catalog number 553057).

### Example 1: Mice with Humanized IL2RA Gene

The mouse IL2RA gene (NCBI Gene ID: 16184, Primary source: MGI: 96549, UniProt ID: P01590) is located at 11642792 to 11693194 of chromosome 2 (NC_000068.7), and the human IL2RA gene (NCBI Gene ID: 3559, Primary source: HGNC: 6008, UniProt ID: P01589) is located at 6010694 to 6062370 of chromosome 10 (NC_000010.11). FIG. 1A shows the mouse transcript NM_008367.3 (SEQ ID NO: 1) and the corresponding protein sequence NP_032393.3 (SEQ ID NO: 2); and FIG. 1B shows the human transcript NM_000417.2 (SEQ ID NO: 3) and the corresponding protein sequence NP_000408.1 (SEQ ID NO: 4).

A gene sequence encoding the human IL33 protein can be introduced into the endogenous mouse IL2RA locus, such that the mouse can express a human or humanized IL2RA protein. Mouse cells can be modified by various gene-editing techniques, for example, replacement of specific mouse IL2RA gene sequences with human IL2RA gene sequences at the endogenous mouse IL2RA locus. For example, under control of a mouse IL2RA regulatory element, a sequence about 8807 bp spanning from exon 2 (including only a part of exon 2) to exon 6 (including only a part of exon 6) of mouse IL2RA gene was replaced with a corresponding human DNA sequence to obtain a humanized IL2RA locus, thereby humanizing mouse IL2RA gene (shown in FIG. 2).

Mouse and human IL2RA DNA were obtained using Bacterial Artificial Chromosome (BAC). As shown in the schematic diagram of the targeting strategy in FIG. 3, the targeting vector has homologous arm sequences upstream and downstream of mouse IL2RA gene locus, and a fragment comprising the human IL2RA gene sequence. The upstream homologous arm sequence (5′ homologous arm, SEQ ID NO: 5) is identical to nucleotide sequence of 11670417-11674302 of NCBI accession number NC_000068.7, and the downstream homologous arm sequence (3′ homologous arm, SEQ ID NO: 6) is identical to nucleotide sequence of 11683554-11689329 of NCBI accession number NC_000068.7. The fragment comprising human IL2RA gene sequence comprises a human genomic IL2RA gene sequence (SEQ ID NO: 7). The human genomic IL2RA gene sequence is identical to nucleotide sequence of 6019444-6026017 of NCBI accession number NC_000010.11.

The connection between the 5′ end of the human IL2RA gene sequence and the mouse IL2RA gene locus was designed as

(SEQ ID NO: 8)
5′-TCTCCTTGCCTTTGCAGAACTGTGTGACGA
TGACCCGCCAGAGATCCCAC-3′,

wherein the last “T” of the sequence “TGTGT” is the last nucleotide of the mouse sequence, and the first “G” of the sequence “GACGA” is the first nucleotide of the human sequence. The connection between the 3′ end of the human IL2RA gene sequence and the mouse IL2RA gene locus was designed as

(SEQ ID NO: 9)
5′-CATGGAGACGTCCATATTTACAACAGAGTA
TAAGGTAGCAGGTGGGCCAG-3′,

wherein the last “A” of the sequence “CAACA” is the last nucleotide of the human sequence, and the first “G” of the sequence “GAGTA” is the first nucleotide of the mouse sequence.

The mRNA sequence of the engineered mouse IL2RA after humanization and its encoded protein sequence are shown in SEQ ID NO: 10 and SEQ ID NO: 11, respectively.

The targeting vector also included an antibiotic resistance gene for positive clone screening (neomycin phosphotransferase gene, or Neo), and two Frt recombination sites flanking the antibiotic resistance gene, that formed a Neo cassette. The Neo cassette is located between exon 6 and exon 7 of mouse IL2RA gene. The connection between the 5′ end of the Neo cassette and the mouse IL2RA gene locus was designed as:

(SEQ ID NO: 12)
5′-GGCAGAGGCCAGCCTGGTCTGCATAGTGAA
TTCCAGTATAGTCGACGGTATCGATAAGCTTGA
TATCGAATTCCGAAGTT-3′,

wherein the last “A” of the sequence “GTATA” is the last nucleotide of the mouse sequence, and the first “G” of the sequence “GTCGA” is the first nucleotide of the Neo cassette. The connection between the 3′ end of the Neo cassette with the mouse IL2RA sequence was designed as

(SEQ ID NO: 13)
5′-TTCATCAGTCAGGTACATAATGGTGGATCC
ACTAGTTCTAGAGCGGCCGCGCCAGAACGACAT
AGTGAGACCTTGTTTCA-3′,

wherein the last “C” of the sequence “GCCGC” is the last nucleotide of the Neo cassette, and the first “G” of the sequence “GCCAG” is the first nucleotide of the mouse sequence. In addition, a coding gene with a negative selectable marker (a gene encoding diphtheria toxin A subunit (DTA)) was also inserted downstream of the 3′ homologous arm of the targeting vector.

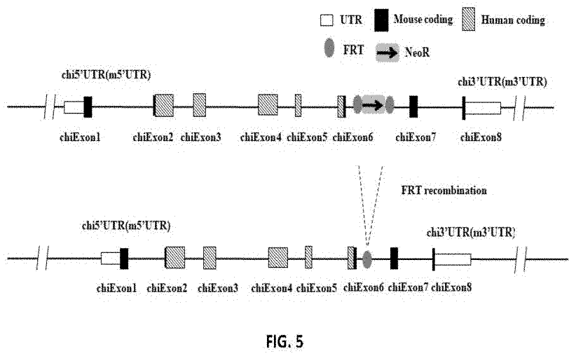

The targeting vector was constructed using standard methods, e.g., by restriction enzyme digestion and ligation, or synthesized directly. The constructed targeting vector sequence was preliminarily verified by restriction enzyme digestion, then verified by sequencing. The correct targeting vector was electroporated and transfected into embryonic stem cells of C57BL/6 mice. The positive selectable marker gene was used to screen the cells, and the integration of exogenous genes was confirmed by PCR and Southern Blot. Specifically, positive clones identified by PCR were further confirmed by Southern Blot (digested with StuI, NdeI, and DraIII, respectively, and then hybridized with 3 probes) to screen out correct positive clone cells. As shown in FIG. 4, the results indicated that among the 11 positive clones (1-H03, 2-A02, 2-007, 2-D10, 2-F09, 2-F11, 3-F02, 3-G11, 3-H03, 3-H07, and 4-B03) confirmed by PCR, all were positive heterozygous clones and no random insertions were detected.

The following primers were used in PCR:

F1:
(SEQ ID NO: 14)
5′-TCTGGTGGAATTTTTGGGGTTCACT-3′;
R1:
(SEQ ID NO: 15)
5′-TCTGTCTCACTCTTTGCTGCAGTTCT-3′;
F2:
(SEQ ID NO: 16)
5′-GCTCGACTAGAGCTTGCGGA-3′;
R2:
(SEQ ID NO: 17)
5′-CTGAGTGGGTCTGAGCAAGATGTCC-3′.

The following probes were used in Southern Blot assays:

5′ probe:
5-F:
(SEQ ID NO: 18)
5′-GATATTCCGGTGTGCCATTCTGCCT-3′,
5-R:
(SEQ ID NO: 19)
5′-GTAGTAGGCAGCGTCAACTCGAAGG-3′;
3′ probe:
3-F:
(SEQ ID NO: 20)
5′-GAAGAGCAGAAGAACCATCTAGCAAG-3′,
3-R:
(SEQ ID NO: 21)
5′-CATTAGTGCTGAGTTTTACTTGGGC-3′;
Neo probe:
Neo-F:
(SEQ ID NO: 22)
5′-GGATCGGCCATTGAACAAGATGG-3′,
Neo-R:
(SEQ ID NO: 23)
5′-CAGAAGAACTCGTCAAGAAGGCG-3′.

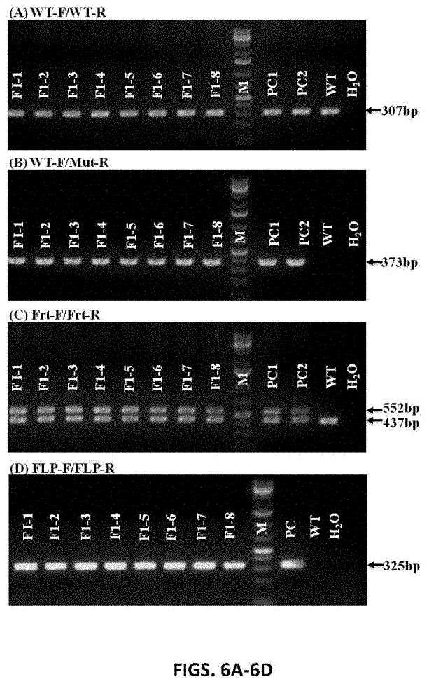

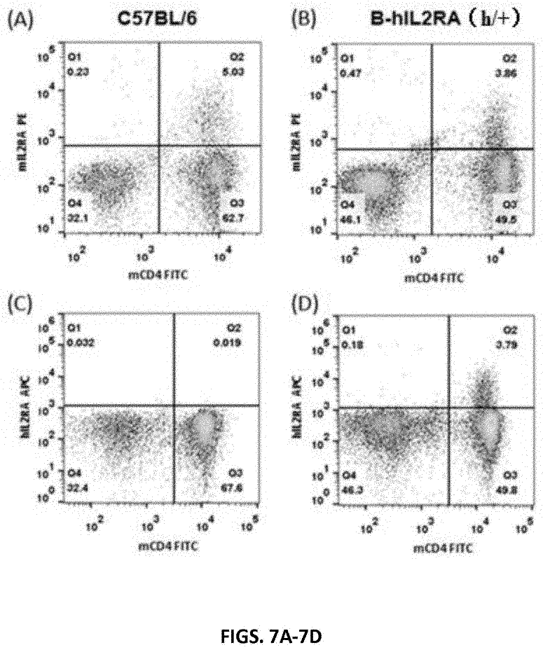

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then mating the F1 generation heterozygous mice with each other. The positive mice were also mated with the Flp transgenic mice to remove the positive selectable marker gene (FIG. 5), and then the humanized IL2RA homozygous mice expressing humanized IL2RA protein were obtained by mating with each other. The genotype of the progeny mice can be identified by PCR. The identification results of exemplary F1 generation mice (Neo cassette-removed) are shown in FIGS. 6A-6D, and mice labelled F1-1, F1-2, F1-3, F1-4, F1-5, F1-6, F1-7, and F1-8 were identified as positive heterozygous clones. The following primers were used in the PCR identification:

WT-F:
(SEQ ID NO: 24)
5′-ACTGATCCCGAGGAGGTAGAGTACG-3′;
WT-R:
(SEQ ID NO: 25)
5′-AGGGTCACACTTACAGTTGCTGGTG-3′;
Mut-R:
(SEQ ID NO: 26)
5′-CTGGGCTCTGTCTCACTCTTTGCTG-3′;
FRT-F:
(SEQ ID NO: 27)
5′-CATGGGTCAGCTGTAGTACGTTGGG-3′;
FRT-R:
(SEQ ID NO: 28)
5′-AGGCTAAATGGGTAGGGAGACTGCT-3′;
Flp-F2:
(SEQ ID NO: 29)
5′-GACAAGCGTTAGTAGGCACATATAC-3′;
Flp-R2:
(SEQ ID NO: 30)
5′-GCTCCAATTTCCCACAACATTAGT-3′.

The results indicated that this method can be used to construct genetically engineered IL2RA mice and the genetic modification can be stably passed to the next generation without random insertions.

### Example 2: Expression of Humanized IL2RA Protein in Mice with Humanized IL2RA Gene

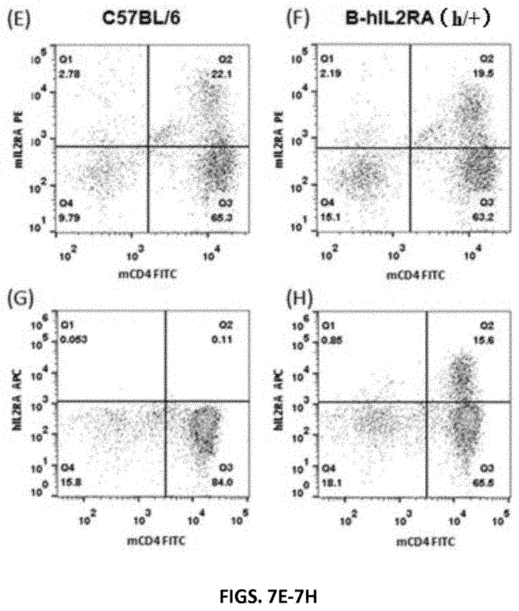

The expression of humanized IL2RA protein in mice was confirmed by experiments. Two 8-week old wild-type C57BL/6 mice and two 8-week old IL2RA gene humanized heterozygous mice (as prepared in Example 1) were selected and randomly placed into two groups (n=2). Mice in one group were stimulated by intraperitoneal injection of an anti-mCD3 antibody (7.5 μg/200 μL). Mice in the other group were not injected with the anti-mCD3 antibody (control group). After 24 hours, mouse spleen cells (splenocytes) were isolated and subjected to flow cytometry analysis. Specifically, T cells were first labelled with PerCP/Cyanine5.5 anti-mouse TCRβ chain (mTCRβ-PerCP/Cy5.5), then stained with either an anti-mouse IL2RA antibody (mCD25 PE) combined with an anti-mouse CD4 antibody (mCD4 FITC), or an anti-human IL2RA antibody (hCD25 APC) combined with the anti-mouse CD4 antibody (mCD4 FITC), followed by flow cytometry analysis. As shown in FIGS. 7A-7H, the results of flow cytometry showed that expression of mouse IL2RA (mIL2RA) were detected in a fraction of CD4+ cells of all mouse spleen cells. Without anti-mCD3 antibody stimulation, the expression level of mIL2RA protein in CD4+ cells in the spleen of wild-type mice and the humanized mice was relatively low. For example, percentages of mCD4+ mIL2RA+ cells in wild-type and the humanized IL2RA mice were 5.03% and 3.86%, respectively (FIG. 7A and FIG. 7B). After anti-mCD3 antibody stimulation, percentages of mCD4+ mIL2RA+ cells in both wild-type and the humanized IL2RA mice showed a significant increase, to 22.1% and 19.5%, respectively (FIG. 7E and FIG. 7F). In addition, regardless of whether being stimulated by anti-mCD3 antibody, expression of humanized IL2RA (hIL2RA) was not detected in the spleen of the wild-type mice (FIG. 7C and FIG. 7G). However, after anti-CD3 antibody stimulation, percentage of mCD4+ hIL2RA+ cells in humanized IL2RA mice increased from 3.79% to 15.6% (FIG. 7D and FIG. 7H). The above results indicate that the in vivo expression level of IL2RA gene in mice is relatively low under normal conditions, and stimulation (e.g., by an anti-CD3 antibody) of CD4+ T cells can induce a high expression of IL2RA gene. In addition, the anti-human IL2RA antibody can detect cells expressing humanized IL2RA protein in the spleen of humanized mice. However, no cells expressing human or humanized IL2RA protein were detected in the spleen of wild-type C57BL/6 wild-type mice

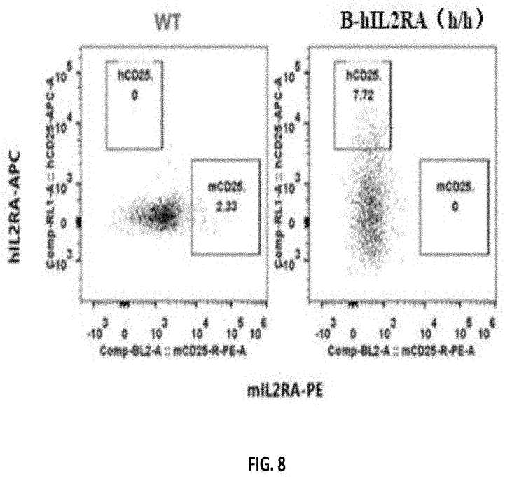

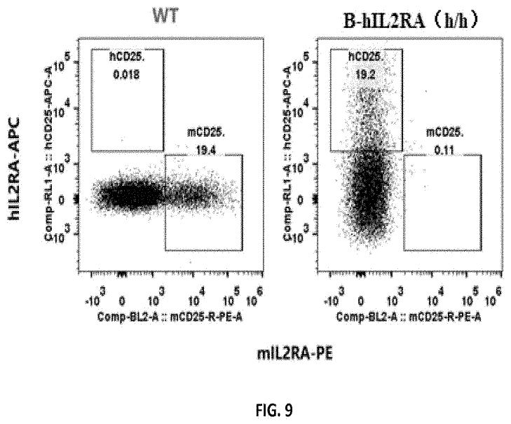

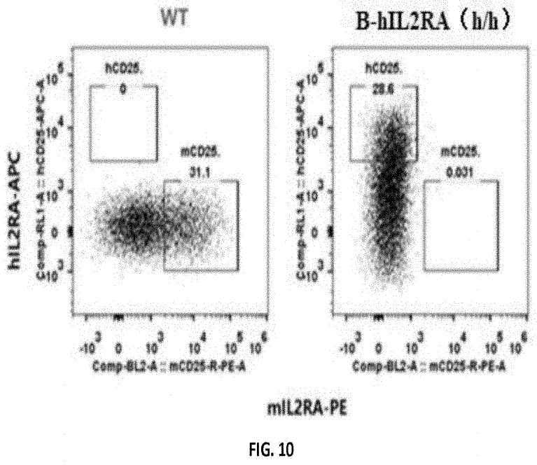

Further, the positive heterozygous mice identified in Example 1 were mated with each other to obtain humanized IL2RA homozygous mice. 10-week old homozygous mice and wild-type C57BL/6 mice (control) were stimulated by an anti-mCD3 antibody for 24 hours. Mouse spleen cells were isolated and subjected to fluorescence-activated cell sorting (FACS) analysis. As shown in FIGS. 8-10, the proportion of T cells and NK cells in the homozygous mice was similar to that in wild-type mice. NK cells, CD4+ T cells and CD8+ T cells (FIGS. 8-10, respectively) all exhibited different levels of IL2RA expression. Specifically, only cells expressing humanized IL2RA protein, but no cells expressing mouse IL2RA protein, were detected in the homozygous mice. In contrast, only cells expressing mouse IL2RA protein, but no cells expressing human or humanized IL2RA protein, were detected in the wild-type mice.

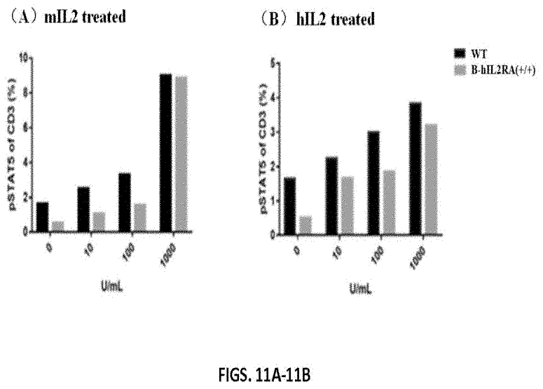

p-STAT5 is a known downstream signal in the IL2/IL2RA-mediated signaling pathway. Thus, the level of p-STAT5 in the IL2RA gene humanized mice can be detected to verify whether the IL2/IL2RA signaling pathway is functional in the humanized mice. Spleen cells from 14-week old wild-type C57BL/6 mice and IL2RA gene humanized homozygous mice were isolated (5×10⁵ cells/well, 4 wells each) and incubated in a 37° C. incubator for 30 minutes. After the incubation, cells were stimulated with recombinant mouse IL2 (mIL2) or human IL2 (hIL2) for 15 minutes at 0 U/mL, 10 U/mL, 100 U/mL, and 1000 U/mL, respectively. Then, the stimulated cells were stained by APC Rat Anti-Mouse CD3 Molecular Complex, permeabilized, and stained by Phospho-Stat5 (Tyr694) (D47E7) XP® Rabbit mAb and Anti-rabbit IgG (H+L), F(ab′)2 Fragment (Alexa Fluor® 488 Conjugate), followed by flow cytometry analysis. As shown in FIGS. 11A-11B, the results shows that after stimulation with mIL2 and hIL2, the level of mouse p-STAT5 was detected in both wild-type C57BL/6 mice and IL2RA gene humanized mouse splenocytes. The results further indicate that the IL2/IL2RA signaling pathway in the humanized mice was functional. In addition, the results indicate that mIL2 can bind to humanized IL2RA protein, and hIL2 can bind to mouse IL2RA protein. The result further shows that the humanized IL2RA can properly interact with mouse IL2RB and IL2RG and transmit the signal into the cells. This is unexpected as the human IL2RA and the mouse IL2RA are only about 60% identical, and the majority of the sequences are humanized.

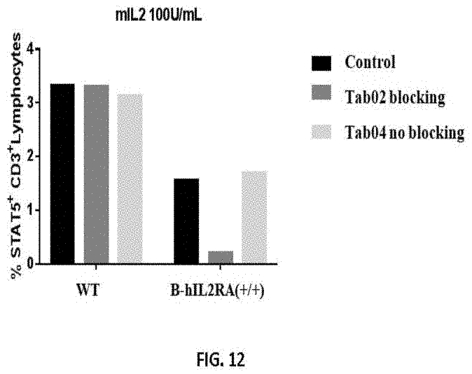

In a separate experiment, anti-human IL2RA antibodies were used to confirm whether the IL2/IL2RA signal pathway was functional. Both Tab02 (daclizumab) and Tab04 (7G7B6) are anti-human IL2RA antibodies. Tab02 is known to be a blocking antibody, while Tab04 can bind to IL2RA but does not exhibit any blocking effects. Spleen cells from wild-type mice and IL2RA gene humanized homozygous mice were isolated (5×10⁵ cells/well, 3 wells each). Cells were first stimulated with mouse IL2 for 15 minutes, then 2 wells (randomly selected) of each cell origin were added with Tab02 and Tab04, respectively (10 μg/mL). One well was added with 10 μg/mL human IgG as a control. The cells were incubated at 37° C. for 30 minutes, and then stained as previously described to detect p-STAT5 by flow cytometry. As shown in FIG. 12, mouse p-STAT5 was detected in all three wells containing cells from wild-type mice. Among the three wells containing cells from IL2RA gene humanized mice, similar levels of mouse p-STAT5 were detected in the control well and the well added with the anti-human IL2RA antibody Tab04. In contrast, the level of p-STAT5 protein was significant reduced in cells from IL2RA gene humanized mice, where the anti-human IL2RA antibody Tab02 was added. The results indicate that the blocking antibody Tab02 can block the IL2/IL2RA signaling pathway by interacting with the humanized IL2RA in the IL2RA gene humanized mice, thereby reducing the level of the p-STAT5 protein. However, Tab02 did not block the IL2/IL2RA signaling pathway in wild-type mice. The results further indicate that the IL2RA gene humanized mice exhibited a functional IL2/IL2RA signaling pathway, and the expressed humanized IL2RA protein can interact with anti-human IL2RA antibody. In addition, the mice prepared by the above method can be used for screening of anti-human IL2RA blocking antibodies and evaluation of drug efficacy.

### Example 3: Pharmacological Validation of IL2RA Gene Humanized Animal Model

The humanized mice prepared by the method described herein can be used to generate various tumor models, which can be used to test drugs (e.g., antibodies) targeting human IL2RA. In one experiment, the IL2RA gene humanized homozygous mice (6-7 week old) were randomly divided into a control group and a treatment group (5 mice in each group) according to mouse body weight. Next day after grouping, each mouse was subcutaneously injected with mouse colon cancer cell MC38 (5×10⁵ cells). The treatment group mice were randomly selected for anti-human IL2RA monoclonal antibody (AB1 and AB2) treatment (10 mg/kg); and the control group mice were injected with an equal amount of control antibodies (same immunoglobulin isotype). The antibodies (i.e., AB1, AB2, and the control antibody) were intraperitoneally injected. The frequency of administration was twice a week (6 times of administrations in total). The tumor volume was measured twice a week and body weight of the mice was recorded as well. Euthanasia was performed when tumor volume of a mouse reached 3000 mm³. The tested antibodies were generated by immunizing mice with human IL2RA proteins. A detailed description of these methods can be found in Murphy, et al., Janeway's Immunobiology. Garland Science, 2016 (9th edition), which is incorporated herein by reference in its entirety.

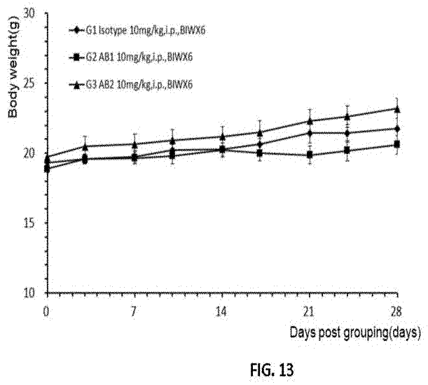

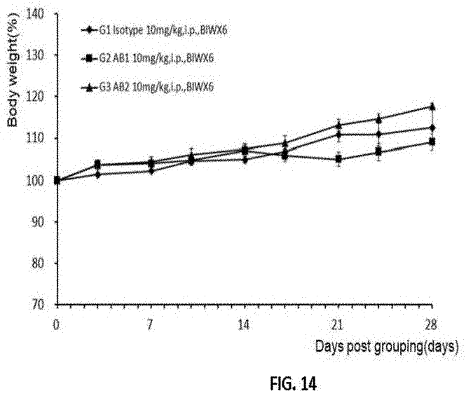

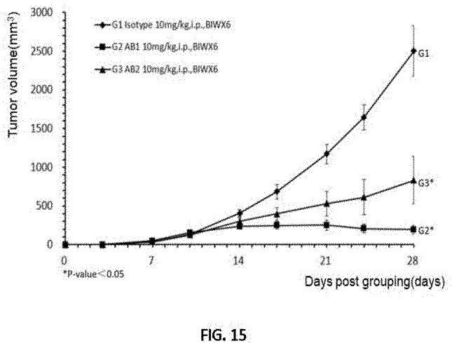

Overall, the animals in each group were healthy. Body weights of all the treatment and control group mice increased, and were not significantly different from each other (FIGS. 13 and 14). The tumor in the control group continued growing during the experimental period (FIG. 15); when compared with the control group mice, the tumor volumes in the treatment groups were smaller than the control group (FIG. 15). Thus, the two anti-human IL2RA antibodies were well tolerated, and the antibodies inhibited the tumor growth in mice. Without wishing to be bound by a particular theory, it is believed that these anti-IL2RA antibodies can target regulatory T cells (Treg) and kill or inhibit the function of Treg cells, as IL2RA is highly expressed on Treg cells, thereby increasing the immune response.

Table 3 shows results for this experiment, including the tumor volumes at Day 10, Day 21, and Day 28 (the last day of the experiment) after the grouping; the survival rate of the mice; the Tumor Growth Inhibition value (TGI_{TV} %); and the statistical differences (P value) in mouse body weights and tumor volume between the treatment and control groups.

TABLE 3

| | | | | | | | P value | |
|---|---|---|---|---|---|---|---|---|
| | | Tumor volume(mm³) | | | | | Body | Tumor |
| | | Day 10 | Day 21 | Day 28 | Survival | TGI_{TV} % | weight | Volume |
| Control | G1 | 141 ± 13 | 1245 ± 126 | 2635 ± 297 | 5/5 | N/A | N/A | N/A |
| Treatment | G2(AB1) | 158 ± 17 | 252 ± 63 | 195 ± 58 | 5/5 | 92.2 | 0.420 | 1.14E-04 |
| groups | G3(AB2) | 128 ± 12 | 531 ± 161 | 833 ± 307 | 5/5 | 66.7 | 0.332 | 0.006 |

At the end of the experiment (day 28), the body weight of each group increased and there was no significant difference between the groups (p>0.05), indicating that the animals tolerated the anti-hIL2RA antibodies (AB1 and AB2) well. With respect to the tumor volume, in the control group (G1), the average tumor volume was 2635±297 mm³. The average tumor volumes in the AB1 treatment group was 195±58 mm³ (G2), and the average tumor volume in the AB2 treatment group was 833±307 mm³ (G3). The tumor volume in each treatment group (G2 and G3) was significantly smaller (P value≤0.05) than that in the control group (G1), with TGI_{TV} % determined as 92.2% and 66.7%, respectively. The results showed that the anti-human IL2RA antibodies AB1 and AB2 both exhibited obvious tumor inhibitory effects (TGI_{TV} %>60%). Specifically, the AB1 antibody exhibited a better tumor inhibitory effects than the AB2 antibody, indicating that different anti-human IL2RA antibodies have different tumor growth inhibitory effects in the IL2RA gene humanized mice. In addition, these antibodies did not exhibit obvious toxic effects in mice.

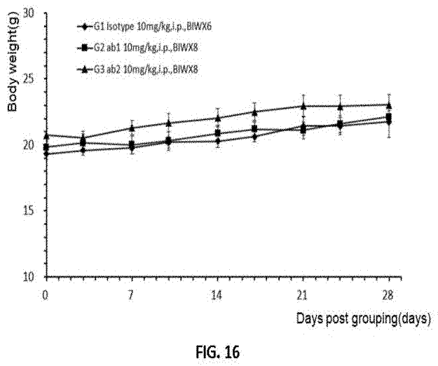

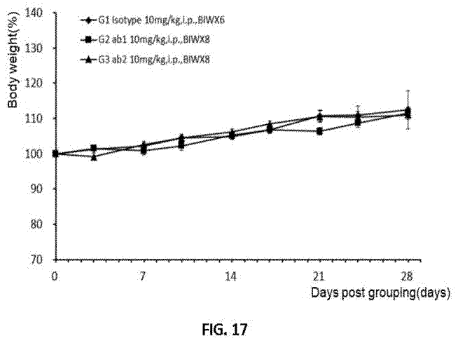

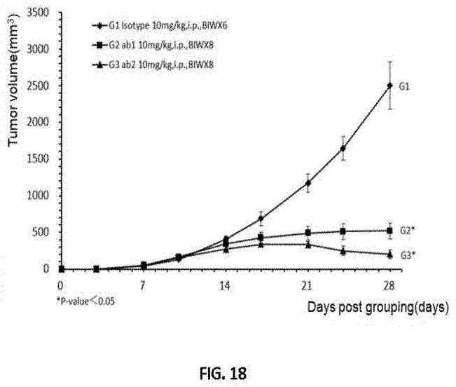

In another experiment, the same method was used to test two different anti-human IL2RA monoclonal antibodies (ab 1 and ab2) to inhibit tumor growth. The results are shown in FIGS. 16-18. Table 4 below shows the results for this experiment. Overall, the animals in each group were healthy. Body weights of all the treatment and control group mice increased, and were not significantly different from each other (FIGS. 16 and 17). As shown in FIG. 18, at the end of the experiment (Day 28), the tumor volume in each treatment group (G2 and G3) was significantly smaller (P value 0.05) than that in the control group (G1). Specifically, the ab2 antibody exhibited a better tumor inhibitory effect than the ab1 antibody.

TABLE 4

| | | | | | | | P value | |
|---|---|---|---|---|---|---|---|---|
| | | Tumor volume(mm³) | | | | | Body | Tumor |
| | | Day 10 | Day 21 | Day 28 | Survival | TGI_{TV} % | weight | Volume |
| Control | G1 | 131 ± 15 | 1171 ± 125 | 2502 ± 325 | 5/5 | N/A | N/A | N/A |
| Treatment | G2(AB1) | 162 ± 24 | 490 ± 93 | 519 ± 111 | 5/5 | 79.3 | 0.787 | 4.16E-04 |
| groups | G3(AB2) | 159 ± 17 | 334 ± 44 | 203 ± 58 | 5/5 | 91.9 | 0.392 | 1.17E-04 |

The above results demonstrated that the IL2RA gene humanized mouse model can be used as an in vivo animal model for screening, evaluating human IL2RA-associated signaling pathway regulators, and testing the efficacy of multiple anti-human IL2RA antibodies.

The non-human mammals described herein can also be prepared through other gene editing systems and approaches, including but not limited to: zinc finger nuclease (ZFN) techniques, transcriptional activator-like effector factor nuclease (TALEN) technique, homing endonuclease (megakable base ribozyme), clustered regularly interspaced short palindromic repeats (CRISPR), or other techniques.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric interleukin-2 receptor alpha chain (IL2RA).

2. The animal of claim 1, wherein the sequence encoding the human or chimeric IL2RA is operably linked to an endogenous regulatory element at the endogenous IL2RA gene locus in the at least one chromosome.

3. The animal of claim 1, wherein the sequence encoding a human or chimeric IL2RA comprises a sequence encoding an amino acid sequence that is at least 70%, 75%, 80%, 85%, 90%, 95%, 99%, or 100% identical to human IL2RA (NP_000408.1 (SEQ ID NO: 4)).

4. The animal of claim 1, wherein the sequence encoding a human or chimeric IL2RA comprises a sequence encoding an amino acid sequence that is at least 70%, 75%, 80%, 85%, 90%, 95%, 99%, or 100% identical to SEQ ID NO: 11.

5. The animal of claim 1, wherein the sequence encoding a human or chimeric IL2RA comprises a sequence that is at least 70%, 75%, 80%, 85%, 90%, 95%, 99%, or 100% identical to amino acids 25-237 of SEQ ID NO: 4.

6. The animal of any one of claims 1-5, wherein the animal is a mammal, e.g., a monkey, a rodent or a mouse.

7. The animal of any one of claims 1-5, wherein the animal is a mouse.

8. The animal of any one of claims 1-7, wherein the animal does not express endogenous IL2RA.

9. The animal of claim 1, wherein the animal has one or more cells expressing human or chimeric IL2RA.

10. The animal of claim 1, wherein the animal has one or more cells expressing human or chimeric IL2RA, and a human IL2 can bind to the expressed human or chimeric IL2RA.

11. The animal of claim 1, wherein the animal has one or more cells expressing human or chimeric IL2RA, and an endogenous IL2 can bind to the expressed human or chimeric IL2RA.

12. A genetically-modified, non-human animal, wherein the genome of the animal comprises a replacement of a sequence encoding a region of endogenous IL2RA with a sequence encoding a corresponding region of human IL2RA at an endogenous IL2RA gene locus.

13. The animal of claim 12, wherein the sequence encoding the corresponding region of human IL2RA is operably linked to an endogenous regulatory element at the endogenous IL2RA locus, and one or more cells of the animal expresses a chimeric IL2RA.

14. The animal of claim 12, wherein the animal does not express endogenous IL2RA.

15. The animal of claim 12, wherein the replaced locus is the extracellular region of IL2RA.

16. The animal of claim 12, wherein the animal has one or more cells expressing a chimeric IL2RA having an extracellular region, a transmembrane region, and a cytoplasmic region, wherein the extracellular region comprises a sequence that is at least 50%, 60%, 70%, 80%, 90%, 95%, or 99% identical to the extracellular region of human IL2RA.

17. The animal of claim 16, wherein the extracellular region of the chimeric IL2RA has a sequence that has at least 10, 20, 30, 40, 50, 60, 70, 80, 90, or 100 contiguous amino acids that are identical to a contiguous sequence present in the extracellular region of human IL2RA.

18. The animal of claim 12, wherein the animal is a mouse, and the sequence encoding the region of endogenous IL2RA is exon 2, exon 3, exon 4, exon 5, and/or exon 6 of the endogenous mouse IL2RA gene.

19. The animal of claim 12, wherein the animal is heterozygous with respect to the replacement at the endogenous IL2RA gene locus.

20. The animal of claim 12, wherein the animal is homozygous with respect to the replacement at the endogenous IL2RA gene locus.

21. A method for making a genetically-modified, non-human animal, comprising: replacing in at least one cell of the animal, at an endogenous IL2RA gene locus, a sequence encoding a region of an endogenous IL2RA with a sequence encoding a corresponding region of human IL2RA.

22. The method of claim 21, wherein the sequence encoding the corresponding region of human IL2RA comprises exon 1, exon 2, exon 3, exon 4, exon 5, exon 6, exon 7, and/or exon 8 of a human IL2RA gene.

23. The method of claim 21, wherein the sequence encoding the corresponding region of IL2RA comprises exon 2, exon 3, exon 4, exon 5, and/or exon 6, or part thereof, of a human IL2RA gene.

24. The method of claim 21, wherein the sequence encoding the corresponding region of human IL2RA encodes amino acids 25-237 of SEQ ID NO: 4.

25. The method of claim 21, wherein the region is located within the extracellular region of IL2RA.

26. The method of claim 21, wherein the animal is a mouse, and the endogenous IL2RA locus is exon 2, exon 3, exon 4, exon 5, and/or exon 6 of the mouse IL2RA gene.

27. A non-human animal comprising at least one cell comprising a nucleotide sequence encoding a chimeric IL2RA polypeptide, wherein the chimeric IL2RA polypeptide comprises at least 50 contiguous amino acid residues that are identical to the corresponding contiguous amino acid sequence of a human IL2RA, wherein the animal expresses the chimeric IL2RA.

28. The animal of claim 27, wherein the chimeric IL2RA polypeptide has at least 50 contiguous amino acid residues that are identical to the corresponding contiguous amino acid sequence of a human IL2RA extracellular region.

29. The animal of claim 27, wherein the chimeric IL2RA polypeptide comprises a sequence that is at least 90%, 95%, or 99% identical to amino acids 25-237 of SEQ ID NO: 4.

30. The animal of claim 27, wherein the nucleotide sequence is operably linked to an endogenous IL2RA regulatory element of the animal.

31. The animal of claim 27, wherein the chimeric IL2RA polypeptide comprises an endogenous IL2RA transmembrane region and/or an endogenous IL2RA cytoplasmic region.

32. The animal of claim 27, wherein the nucleotide sequence is integrated to an endogenous IL2RA gene locus of the animal.

33. The animal of claim 27, wherein the chimeric IL2RA has at least one mouse IL2RA activity and/or at least one human IL2RA activity.

34. A method of making a genetically-modified mouse cell that expresses a chimeric IL2RA, the method comprising:
replacing at an endogenous mouse IL2RA gene locus, a nucleotide sequence encoding a region of mouse IL2RA with a nucleotide sequence encoding a corresponding region of human IL2RA, thereby generating a genetically-modified mouse cell that includes a nucleotide sequence that encodes the chimeric IL2RA, wherein the mouse cell expresses the chimeric IL2RA.

35. The method of claim 34, wherein the chimeric IL2RA comprises:
an extracellular region of human IL2RA comprising a human signal peptide sequence; and
a transmembrane and/or a cytoplasmic region of mouse IL2RA.

36. The method of claim 35, wherein the nucleotide sequence encoding the chimeric IL2RA is operably linked to an endogenous IL2RA regulatory region, e.g., promoter.

37. The animal of any one of claims 1-20 and 27-33, wherein the animal further comprises a sequence encoding an additional human or chimeric protein.

38. The animal of claim 37, wherein the additional human or chimeric protein is programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α(SIRPα) or TNF Receptor Superfamily Member 4 (OX40).

39. The method of any one of claims 21-26 and 34-36, wherein the animal or mouse further comprises a sequence encoding an additional human or chimeric protein.

40. The method of claim 39, wherein the additional human or chimeric protein is PD-1, CTLA-4, LAG-3, BTLA, PD-L1, CD27, CD28, CD47, CD137, CD154, TIGIT, TIM-3, GITR, SIRPa or OX40.

41. A method of determining effectiveness of an anti-IL2RA antibody for the treatment of cancer, comprising:
administering the anti-IL2RA antibody to the animal of any one of claims 1-20 and 27-33, wherein the animal has a tumor; and
determining the inhibitory effects of the anti-IL2RA antibody to the tumor.

42. The method of claim 41, wherein the tumor comprises one or more cells that express IL2RA.

43. The method of claim 41, wherein the tumor comprises one or more cancer cells that are injected into the animal.

44. The method of claim 41, wherein determining the inhibitory effects of the anti-IL2RA antibody to the tumor involves measuring the tumor volume in the animal.

45. The method of claim 41, wherein the tumor cells are melanoma cells, leukemias, lymphomas, solid tumor cells, colorectal cancer cells, ovarian cancer cells, prostate cancer cells, melanoma cells, lung cancer cells, breast cancer cells, gastric cancer cells, esophageal squamous cell carcinoma (ESCC) cells, and/or head-and-neck cancer cells.

46. A method of determining effectiveness of an anti-IL2RA antibody and an additional therapeutic agent for the treatment of a tumor, comprising
administering the anti-IL2RA antibody and the additional therapeutic agent to the animal of any one of claims 1-20 and 27-33, wherein the animal has a tumor; and
determining the inhibitory effects on the tumor.

47. The method of claim 46, wherein the animal further comprises a sequence encoding a human or chimeric programmed cell death protein 1 (PD-1).

48. The method of claim 46, wherein the animal further comprises a sequence encoding a human or chimeric programmed death-ligand 1 (PD-L1).

49. The method of claim 46, wherein the additional therapeutic agent is an anti-PD-1 antibody or an anti-PD-L1 antibody.

50. The method of claim 46, wherein the tumor comprises one or more tumor cells that express IL2RA, PD-1 or PD-L1.

51. The method of claim 46, wherein the tumor is caused by injection of one or more cancer cells into the animal.

52. The method of claim 46, wherein determining the inhibitory effects of the treatment involves measuring the tumor volume in the animal.

53. The method of claim 46, wherein the animal has acute lymphoblastic leukemia (ALL), B-cell chronic lymphocytic leukemia (B-CLL), hairy cell leukemia (HCL), solid tumors, colorectal cancer, ovarian cancer, prostate cancer, melanoma, lung cancer, breast cancer, gastric cancer, esophageal squamous cell carcinoma (ESCC), leukemia, lymphoma, multiple myeloma, sarcoma, and/or head-and-neck cancer.

54. A method of determining effectiveness of an anti-IL2RA antibody for treating an autoimmune disorder, comprising:
a) administering the anti-IL2RA antibody to the animal of any one of claims 1-20 and 27-33, wherein the animal has the autoimmune disorder; and
b) determining effects of the anti-IL2RA antibody for treating the auto-immune disease.

55. The method of claim 54, wherein the autoimmune disorder is multiple sclerosis, rheumatoid arthritis, juvenile idiopathic arthritis, type 1 diabetes, aplastic anemia, asthma, idiopathic thrombocytopenia, uveitis, and eczema.

56. A protein comprising an amino acid sequence, wherein the amino acid sequence is one of the following:
(a) an amino acid sequence set forth in SEQ ID NO: 11;
(b) an amino acid sequence that is at least 90% identical to SEQ ID NO: 11;
(c) an amino acid sequence that is at least 91%, 92%, 93%, 94%, 95%, 96%, 97%, 98%, or 99% identical to SEQ ID NO: 11;
(d) an amino acid sequence that is different from the amino acid sequence set forth in SEQ ID NO: 11 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 11.

57. A nucleic acid comprising a nucleotide sequence, wherein the nucleotide sequence is one of the following:
(a) a sequence that encodes the protein of claim 54;
(b) SEQ ID NO: 10;
(c) a sequence that is at least 90% identical to SEQ ID NO: 10;
(d) a sequence that is at least 91%, 92%, 93%, 94%, 95%, 96%, 97%, 98%, or 99% identical to SEQ ID NO: 10; and
(e) a sequence that is at least 91%, 92%, 93%, 94%, 95%, 96%, 97%, 98%, or 99% identical to SEQ ID NO: 10.

58. A cell comprising the protein of claim 56 and/or the nucleic acid of claim 57.

59. An animal comprising the protein of claim 56 and/or the nucleic acid of claim 57.
